(19) United States
(12) Patent Application Publication
(10) Pub. No.: US 2020/0405858 A1
(43) Pub. Date: Dec. 31, 2020

(54) MODULATION OF BIOPHOTONIC REGIMENS

(71) Applicants: Giacomo ROSSI, Matelica (IT); Remigio PIERGALLINI, Grottammare (IT); Nikolaos LOUPIS, Athens (GR); Angela PALUMBO PICCIONELLO, Matelica (GR); Andrea MARCHEGIANI, Matelica (IT); Andrea SPATERNA, Matelica (IT); Alberto SALVAGGIO, Matelica (IT); David OHAYON, Dollard-Des-Ormeaux (CA); KLOX TECHNOLOGIES LIMITED, Dublin (IE)

(72) Inventors: Giacomo ROSSI, Matelica (IT); Remigio PIERGALLINI, Grottammare (IT); Nikolaos LOUPIS, Athens (GR); Angela PALUMBO PICCIONELLO, Matelica (IT); Andrea MARCHEGIANI, Matelica (IT); Andrea SPATERNA, Matelica (IT); Alberto SALVAGGIO, Matelica (IT); David OHAYON, Dollard-Des-Ormeaux (CA)

(21) Appl. No.: 16/764,678

(22) PCT Filed: Nov. 16, 2018
(86) PCT No.: PCT/CA2018/051464

§ 371 Date: May 15, 2020

Related U.S. Application Data
(60) Provisional application No. 62/587,820, filed on Nov. 17, 2017.
(60) Provisional application No. 62/680,952, filed on Jun. 5, 2018.
(60) Provisional application No. 62/701,248, filed on Jul. 20, 2018.

(51) Int. Cl. A61K 41/00; A61N 5/06

(52) U.S. Cl. CPC A61K 41/0057; A61N 2005/0626; A61N 5/062

(57) ABSTRACT

The present disclosure generally relates to methods and processes for modulating biophotonic regimens as well as to the modulated biophotonic regimens resulting therefrom. The present disclosure also generally relates to biophotonic methods for stimulating energy production in a tissue.

### CROSS-REFERENCE TO RELATED APPLICATION

This application claims the benefit of and priority to U.S. provisional patent application No. 62/587,820, filed on Nov. 17, 2017; to U.S. provisional patent application No. 62/680,952, filed on Jun. 5, 2018; and to U.S. provisional patent application No. 62/701,248, filed on Jul. 20, 2018; the content of all of which is herein incorporated in entirety by reference.

### FIELD OF TECHNOLOGY

The present disclosure generally relates to methods and processes for modulating biophotonic regimens as well as to the modulated biophotonic regimens resulting therefrom. The present disclosure also generally relates to biophotonic methods for stimulating energy production in a tissue.

### BACKGROUND INFORMATION

Exposure to light is known to modulate the activity of some cellular tissues. Different wavelengths of light act on different mechanisms within individual cells of cellular tissues to stimulate or suppress biological activity within the cells in a process commonly referred to as photobiomodulation (PBM).

Photobiomodulation is a general result of light therapy or phototherapy which uses different wavelengths of light to, inter alia, promote healing of tissues (e.g., wound healing), revitalize and rejuvenate tissues (e.g., skin rejuvenation) and cells, and in some circumstances, stimulate cellular regeneration and regrowth. It is generally accepted that some cellular activities can be up-regulated and/or down-regulated by specific wavelengths of light.

Biological organisms comprise numerous molecules that can act as endogenous light-absorbing molecules. Examples of such endogenous light-absorbing molecules include, but are not limited to, molecules such as cytochrome-C oxidase, hemoglobin, myoglobin, porphyrins, porphyrin-like molecules, vitamins, and nicotinamide adenine dinucleotide (NADH), found in cellular tissues that act as photon acceptors. They react to light and serve to initiate biochemical cellular in responses to photons. Exposure of cellular tissues to light is also known to affect mitochondrial density and activity, cell proliferation and adhesiveness, and DNA and RNA production. Phototherapy has been shown to affect vascular endothelial growth factor (VEGF) expression (both enhancement and suppression) and to protect against a wide variety of toxins, such as chemical, ionizing, and toxicological insults. At least some of the known effects of the various wavelengths on body tissues are as follows. Light in the yellow range (approximately 577 nm to 597 nm) has been shown to switch off collagenase production by down-regulating MMPs and switching on new collagen production. Collagenases are enzymes that break down the native collagen that holds animal tissue together. Thus, use of light in the yellow range for phototherapy ultimately results in increased cohesion of cells in animal tissue. Light in the red range (approximately 640 nm to 700 nm) has been shown to decrease inflammation in injured tissue, increase ATP production, and otherwise stimulate beneficial cellular activity. Light in the blue range (approximately 405 nm to 450 nm) has been shown to kill various microorganisms.

However, phototherapy may have undesired and/or harmful effects on cellular activities and processes if the parameters of phototherapy (e.g., wavelength, power density of light, period of illumination, or the like) are not monitored and/or not controlled.

As such, there is a need in the art for methods and processes that allow monitoring and/or assessing of the efficiency and/or progression of a biophotonic regimen based on the activity of the cellular processes affected by exposure to light. In addition, there is a need to be able to optimize and/or individualize (i.e., tailor) a biophotonic regimen to suite the requirements of a subject in need of or undergoing a biophotonic regimen based on an ability to monitor fluctuations in cellular and tissue levels of energy production.

### SUMMARY OF DISCLOSURE

In accordance with various aspects, the present disclosure provides for a method for modulating a biophotonic treatment of a subject, the method comprising: a) assaying a level of energy production in tissue under biophotonic treatment; b) comparing the level of energy production obtained in a) with a level of energy production in non-treated tissue, and c) wherein when the level of energy production obtained in a) is lower than the level of energy production obtained in b) varying the parameters of the biophotonic treatment.

In accordance with various aspects, the present disclosure provides for a method for modulating a biophotonic treatment of a subject, the method comprising: a) assaying a first level of energy production in tissue under biophotonic treatment; b) comparing the first level of energy production obtained in a) with a second level of energy production in non-treated tissue, and c) wherein when the first level of energy production obtained in a) is lower than the second level of energy production obtained in b) varying the parameters of the biophotonic treatment.

In accordance with various aspects, the present disclosure provides for a method for modulating a biophotonic treatment of a subject, the method comprising: a) assaying the levels of energy production in tissue under biophotonic treatment at a first time point during the biophotonic treatment; b) assaying levels of energy production in tissue under biophotonic treatment at a second time point during the biophotonic treatment; b) comparing the levels of energy production obtained in a) with the levels of energy production in tissue of the subject not under biophotonic treatment, wherein when the levels of energy production obtained in a) are lower than the levels of energy production obtained in b); and c) varying the parameters of the biophotonic treatment.

In accordance with various aspects, the present disclosure provides for a method for modulating the efficiency of a biophotonic regimen at healing a tissue or at modulating any biological-based condition (caused by injury or not) in a subject in need of biophotonic therapy; the method comprising: a) measuring the level of ATP production in the wounded tissue prior to commencement of the biophotonic regimen; b) measuring the level of ATP production in the wounded tissue after commencement of the biophotonic regimen; wherein when the level of ATP production in b) is lower than the level of ATP production in a), parameters of the biophotonic regimen are modulated.

In accordance with various aspects, the present disclosure provides for a method for stimulating mitochondrial biogenesis in a tissue, the method comprising applying a biophotonic composition to the tissue; and illuminating the applied biophotonic composition for a time sufficient to activate the biophotonic composition.

In accordance with various aspects, the present disclosure provides for a method for modulating a biophotonic treatment of a subject, the method comprising: a) assaying a level of energy production in tissue of the subject under biophotonic treatment; b) comparing the level of energy production obtained in a) with the level of energy production in non-treated tissue, and c) wherein when the level of energy production obtained in a) are lower than the level of energy production obtained in b), varying the parameters of the biophotonic treatment.

In accordance with various aspects, the present disclosure provides for a method for modulating the efficiency of a biophotonic regimen at healing a wounded tissue; the method comprising: a) measuring the level of at least one cellular marker associated with energy production in the wounded tissue prior to commencement of the biophotonic regimen; b) measuring the level of at least one cellular marker associated with energy production in the wounded tissue after commencement of the biophotonic regimen; wherein when the level of the at least one cellular marker associated with energy production in b) is lower than the level of the at least one cellular marker associated with energy production in a), parameters of the biophotonic regimen are modulated; wherein the at least one cellular marker associated with energy production is selected from ATP, ADP, GTP, GDP, Hsp70, Hsp60, MMPs, leptins, UCPs, ATP synthase, NADH, NAD, FAD, FADH, pyruvate, succinate, fumarate, co-enzyme A, pyruvate dehydrogenase, acetyl-CoA, citrate synthase, citrate, aconitase, isocitrate dehydrogenase, alpha-ketogluterate dehydrogenase, succinyl-CoA, succinyl CoC dehydrogenase, succinate dehydrogenase, fumarase, malate, malate dehydrogenase, oxalo acetate, citric acid, NADH-coenzyme Q oxidoreductase, succinate-Q oxidoreductase, flavoprotein-Q oxidoreductase, Q-cytochrome c oxidoreductase, cytochrome c and cytochrome c oxidase.

In accordance with various aspects, the present disclosure provides for a method for modulating the efficiency of a biophotonic regimen at healing an inflamed tissue; the method comprising: a) measuring the level of at least one cellular marker associated with energy production in the inflamed tissue prior to commencement of the biophotonic regimen; b) measuring the level of at least one cellular marker associated with energy production in the inflamed tissue after commencement of the biophotonic regimen; wherein when the level of the at least one cellular marker associated with energy production in b) is lower than the level of the at least one cellular marker associated with energy production in a), parameters of the biophotonic regimen are modulated; wherein the at least one cellular marker associated with energy production is selected from ATP, ADP, GTP, GDP, Hsp70, Hsp60, MMPs, leptins, UCPs, ATP synthase, NADH, NAD, FAD, FADH, pyruvate, succinate, fumarate, co-enzyme A, pyruvate dehydrogenase, acetyl-CoA, citrate synthase, citrate, aconitase, isocitrate dehydrogenase, alpha-ketogluterate dehydrogenase, succinyl-CoA, succinyl CoC dehydrogenase, succinate dehydrogenase, fumarase, malate, malate dehydrogenase, oxalo acetate, citric acid, NADH-coenzyme Q oxidoreductase, succinate-Q oxidoreductase, flavoprotein-Q oxidoreductase, Q-cytochrome c oxidoreductase, cytochrome c and cytochrome c oxidase.

In accordance with various aspects, the present disclosure provides for a method for modulating the efficiency of a biophotonic regimen at healing an infected tissue; the method comprising: a) measuring the level of at least one cellular marker associated with energy production in the infected tissue prior to commencement of the biophotonic regimen; b) measuring the level of at least one cellular marker associated with energy production in the infected tissue after commencement of the biophotonic regimen; wherein when the level of the at least one cellular marker associated with energy production in b) is lower than the level of the at least one cellular marker associated with energy production in a), parameters of the biophotonic regimen are modulated; wherein the at least one cellular marker associated with energy production is selected from ATP, ADP, GTP, GDP, Hsp70, Hsp60, MMPs, leptins, UCPs, ATP synthase, NADH, NAD, FAD, FADH, pyruvate, succinate, fumarate, co-enzyme A, pyruvate dehydrogenase, acetyl-CoA, citrate synthase, citrate, aconitase, isocitrate dehydrogenase, alpha-ketogluterate dehydrogenase, succinyl-CoA, succinyl CoC dehydrogenase, succinate dehydrogenase, fumarase, malate, malate dehydrogenase, oxalo acetate, citric acid, NADH-coenzyme Q oxidoreductase, succinate-Q oxidoreductase, flavoprotein-Q oxidoreductase, Q-cytochrome c oxidoreductase, cytochrome c and cytochrome c oxidase.

In accordance with various aspects, the present disclosure provides for a method for modulating the efficiency of a biophotonic regimen at healing a wounded tissue; the method comprising: a) measuring the level of at least one citric acid cycle-associated molecule in the wounded tissue prior to commencement of the biophotonic regimen; b) measuring the level of the at least one citric acid cycle-associated molecule in the wounded tissue after commencement of the biophotonic regimen; wherein when the level of the at least one citric acid cycle-associated molecule in b) is lower than the level of the at least one citric acid cycle-associated molecule in a), parameters of the biophotonic regimen are modulated; wherein the at least one citric acid cycle-associated molecule is selected from coenzyme A, citrate, aconitase, isocitrate, alpha-ketoglutarate, succinyl-CoA, succinate, fumarate, malate, oxaloacetate, pyruvate, acetyl-CoA, aconitase, isocitrate dehydrogenase, alpha-ketoglutarate dehydrogenease, GDP, NAD, FAD, succinyl-CoA synthetase, succinic dehydrogenase fumarase, malate dehydrogenase, citrate synthase, pyruvate carboxylase, and pyruvate dehydrogenase.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of the structure of the mitochondrial respiratory chain in eukaryotic cells.

FIG. 2 is a schematic representation of anabolic pathways that promote cell growth.

FIG. 3 shows pictures from a histology assessment of samples of an incision wound taken from a treated area (A) and an untreated area (B). A1 and B1: Hematoxylin Eosin (H&E) coloration. A2 and B2: Immunoblotting for Pan-Cytokeratin. A3 and B3: Collagen Immunograde III. A4 and B4: Semi-Chemically Coloration with Masson Tricromic.

FIGS. 4A-4C are transmission electron micrographs showing cells of canine skin biopsy samples taken from a pyoderma site treated with a biophotonic regimen according to one embodiment of the present disclosure at time T0 (FIG. 4A) and at time T1 (FIG. 4B) as well as a control (untreated skin) at time T1 (FIG. 4C).

### DETAILED DISCLOSURE

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “subject” refers to a human subject or to an animal subject. In some instances, the term “subject” may refer to a plant subject.

The term “biophotonic” as used herein refers to the generation, manipulation, detection and application of photons in a biologically relevant context. As used herein, the expression “biophotonic composition” refers to a composition as described herein that may be activated by light to produce photons for biologically relevant applications. As used herein, the expression “biophotonic regimen” or “biophotonic treatment” or “biophotonic therapy” refers to the use of a combination of a biophotonic composition as defined herein and an illumination period of that biophotonic composition to activate the biophotonic composition.

The term “topical” means as applied to body surfaces, such as the skin, mucous membranes, vagina, oral cavity, external or internal wound or surgical wound sites, and the like.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation, is capable of absorbing the light. The light-absorbing molecules readily undergo photoexcitation and in some instances can then transfer its energy to other molecules or emit it as light.

The term “gels” as used herein refers to substantially dilute cross-linked systems. Gels may be semi-solids and exhibit substantially no flow when in the steady state at room temperature (e.g. about 20° C.-25° C.). By steady state is meant herein during a treatment time and under treatment conditions. Gels, as defined herein, may be physically or chemically cross-linked. As defined herein, gels also include gel-like compositions such as viscous liquids.

The term “membrane” as used in the expression “biophotonic membrane” refers to a biophotonic composition which is in the form of a biophotonic membrane containing at least one light-absorbing molecule. The biophotonic membranes of the present disclosure may be deformable. They may be elastic or non-elastic (i.e. flexible or rigid). The biophotonic membrane, for example, may be in a peel-off form (‘peelable’) to provide ease and speed of use. In certain instances, the tear strength and/or tensile strength of the peel-off form is greater than its adhesion strength. This may help handleability of the biophotonic membrane. In some instances, the biophotonic membrane comprises silicone. In some instances, the biophotonic membrane comprises a thermogelling solution.

The expression “actinic light” as used herein refers to light energy emitted from a specific light source (e.g., lamp, LED, or laser) and capable of being absorbed by matter (e.g., the light-absorbing molecule defined above). In some embodiments, the actinic light is visible light.

As used herein, the term “treated” in expressions such as: “treated skin” and “treated area/portion of the skin”, “treated tissue” and “treated soft tissue”, refers to a skin, a tissue or a soft tissue surface or layer(s) onto which a method according to the embodiments of the present disclosure has been performed. For example, in some instances, a treated tissue refers to a tissue onto which the composition of the present disclosure has been applied and which has been illuminated as outlined herein.

In some aspects of these embodiments, the expression “biological tissue” refers to any organ and tissue of a living system or organism. Examples of biological tissue include, but are not limited to: brain, the cerebellum, the spinal cord, the nerves, blood, lungs, heart, blood vessels, skin, hair, fat, nails, bones, cartilage, ligaments, tendons, ovaries, fallopian tubes, uterus, vagina, mammary glands, testes, vas deferens, seminal vesicles, prostate, salivary glands, esophagus, stomach, liver, gallbladder, pancreas, intestines, rectum, anus, kidneys, ureters, bladder, urethra, the pharynx, larynx, bronchi, lungs, diaphragm, hypothalamus, pituitary gland, pineal body or pineal gland, thyroid, parathyroid, adrenals (e.g., adrenal glands), lymph nodes and vessels, skeletal muscles, smooth muscles, cardiac muscle, brain, spinal cord, peripheral nervous system, ears, eyes, nose, and the like.

In other aspects of these embodiments, the expression “biological tissue” refers to individual cells or a population or a group of cells. In some instances, the cells are ex vivo cells. In some other instances, the cells are in vivo. In some other instances, the cells are in vitro.

As used herein, the term “photobiomodulation” also known as low energy photon therapy (LEPT), also known as low energy, low level, low intensity laser therapy, is the area of photomedicine where the ability of monochromatic light to alter cellular function and enhance healing non-destructively is a basis for the treatment of dermatological, musculoskeletal, soft tissue and neurological conditions.

As used herein, the expression “cellular processes” refers to processes that are carried out at the cellular level, but are not necessarily restricted to a single cell. For example, cell communication occurs among more than one cell, but occurs at the cellular level.

As used herein, the expression “mitochondrial biogenesis” refers to processes of growth, amplification and healthy maintenance of the mitochondria. Mitochondrial biogenesis is a complex process involving both nuclear and mitochondrial compounds. Mitochondrial DNA encodes a small number of proteins, which are translated on mitochondrial ribosomes. Most of these proteins are highly hydrophobic subunits of the respiratory chain, which is localized in the inner mitochondrial membrane. Nuclear-encoded proteins are translated on cytosolic ribosomes and imported into mitochondria. These proteins include structural proteins, enzymes or enzyme subunits, components of the import-, replication-, transcription- and translation-machinery and chaperones. Cells have to switch to the less efficient anaerobic energy metabolism, once the capacity for the aerobic respiration (electron chain) does not suffice anymore. It follows that increased mitochondrial biogenesis improves the capacity for aerobic energy metabolism, and thus increases the capacity for an efficient energy production. “Mitochondrial biogenesis” as used throughout this disclosure includes all processes involved in maintenance and growth of the mitochondria, including those required for mitochondrial division and segregation during the cell cycle. Cellular markers associated with mitochondrial biogenesis may include, but are not limited to: Hsp70, Hsp60, TOM, TIM, PAM, SAM, PGC-1alpha, PGC-1beta, ATP synthase, COX subunits, NRF-1, NRF-2, eNOS, SIRTs, TORCs, AMPK, CaMKIV, NO, guanylate cyclase, cGMP, calcineurin, p38 MAPK, RIP140, Sin3A, NADH, and FADH₂. The levels and/or activity of these cellular markers may be measured in order to evaluate and/or assessed mitochondria biosynthesis.

As used herein, the expression “biogenesis-inducing amount” means that the overall mitochondrial biogenesis is at least maintained at the level which was present before the commencement of the biophotonic regimen. This can be determined in vitro by monitoring the amount and state of mitochondrial functioning in a tissue sample. Additionally, this can be determined in vivo by measuring the ATP content or NADH content of tissue; or the oxygen consumption during exercise (VO₂ max), or ex vivo by transcriptomics analysis for upregulation of mitochondrial markers (such as T_{fam}), or by detecting the increased presence of mitochondrial DNA in tissue biopsies.

As used herein, the expression “mitochondrial-stimulating” means that the biophotonic regiment applied to the mitochondria leads to mitochondrial biogenesis and/or to increased ATP production in the cell; an increased capacity for energy production in the cell; an increased capacity for aerobic energy generation or production in the cell; and/or an increased capacity for fat burning.

Without wishing to be bound to any specific theory, embodiments of the present technology have been developed based on the developers' realization that in the context of using biophotonic regimens for the treatment of skin (e.g., healing of a wound), treatment/healing of the skin involves up-regulation of energy production by the treated tissue which coincides with an up-regulation of some cellular markers involved in energy production in the cells of the treated tissue. In particular, the discoverers have observed an increase in the number of mitochondria in the cells of skin tissue undergoing a biophotonic treatment. These findings led the discoverers to propose that cellular processes involved in energy production may be influenced by the parameters of the biophotonic regimen such as, for example, wavelength of the light, power density of the light, type and concentration of light-absorbing molecules, duration of illumination period and of the biophotonic treatment.

These observations make it possible to assess the efficiency of a given a biophotonic regimen. In particular, these findings open the possibility of evaluating/assessing the efficiency of a biophotonic regimen at, for example, treating/healing skin (e.g., healing a wound) in real-time (i.e., during the course of a biophotonic regimen) by assessing the activity of the cellular processes involved in energy production such as mitochondrial biogenesis (e.g., number of mitochondria, size of mitochondria, energy production, number of cristae, localization of mitochondria within the cytoplasm of cells, localization of the mitochondria with respect to other organelles found in the cytoplasm of cell, or the like). This assessment allows modulating/adjusting the parameters of the biophotonic regimen so as to optimize the treatment and the healing process.

In eukaryotic cells, cellular energy production mainly occurs in mitochondria. Mitochondria are thought to be a likely site for the initial effects of light, leading to increased ATP production, modulation of reactive oxygen species, and induction of transcription factors. These effects in turn lead to increased cell proliferation and migration, modulation in levels of cytokines, growth factors and inflammatory mediators, and increased tissue oxygenation. The results of these biochemical and cellular changes in animals and humans include such benefits as increased healing of wounds, pain reduction in arthritis and neuropathies, and amelioration of damage after heart attacks, stroke, nerve injury, brain function, and retinal toxicity.

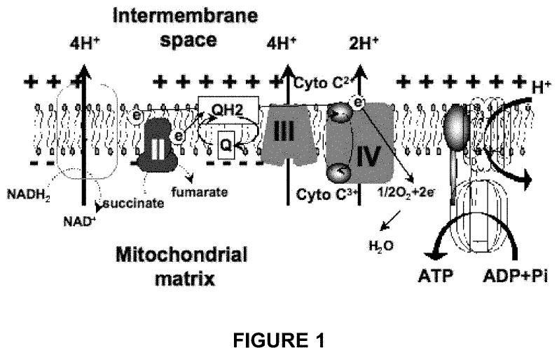

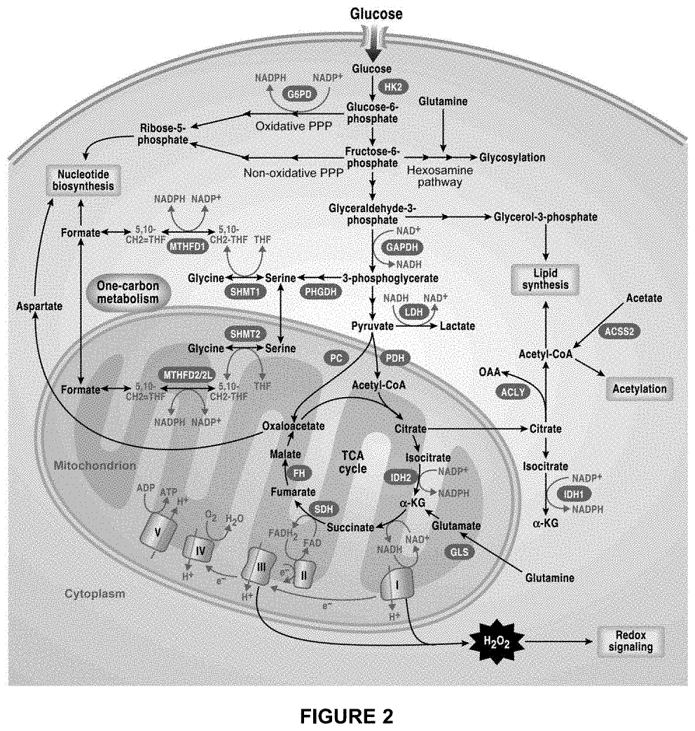

The inner mitochondrial membrane contains 5 complexes of integral membrane proteins: NADH dehydrogenase (Complex I), succinate dehydrogenase (Complex II), cytochrome c reductase (Complex III), cytochrome c oxidase (Complex IV), ATP synthase (Complex V), and two freely diffusible molecules, ubiquinone and cytochrome c, which shuttle electrons from one complex to the next (FIG. 1). The respiratory chain accomplishes the stepwise transfer of electrons from NADH and FADH₂ (produced in the citric acid or Krebs cycle (FIG. 2)) to oxygen molecules to form (with the aid of protons) water molecules harnessing the energy released by this transfer to the pumping of protons (H⁺) from the matrix to the intermembrane space. The gradient of protons formed across the inner membrane by this process of active transport forms a miniature battery. The protons can flow back down this gradient, re-entering the matrix, only through another complex of integral proteins in the inner membrane, the ATP synthase complex.

Absorption spectra obtained for cytochrome c oxidase in different oxidation states were recorded and found to be very similar to the action spectra for biological responses to light. Therefore, it was proposed that cytochrome c oxidase (Cox) is the primary photoacceptor for the red-NIR range in mammalian cells. The single most important molecule in cells and tissue that absorbs light between 630 and 900 nm is Cox (responsible for more than 50% of the absorption greater than 800 nm). Cytochrome C oxidase contains two iron centers, haem a and haem a3 (also referred to as cytochromes a and a3), and two copper centers, CuA and CuB. Fully oxidized cytochrome c oxidase has both iron atoms in the Fe(III) oxidation state and both copper atoms in the Cu(II) oxidation state, while fully reduced cytochrome c oxidase has the iron in Fe(II) and copper in Cu(I) oxidation states. There are many intermediate mixed-valence forms of the enzyme and other coordinate ligands such as CO, CN, and formate can be involved.

The absorption of photons by molecules leads to electronically excited states, and consequently can lead to an acceleration of electron transfer reactions. More electron transport necessarily leads to the increased production of ATP. The light-induced increase in ATP synthesis and increased proton gradient leads to an increasing activity of the Na⁺/H⁺ and Ca²⁺/Na⁺ antiporters, and of all the ATP driven carriers for ions, such as Na⁺/K⁺ ATPase and Ca²⁺ pumps. ATP is the substrate for adenyl cyclase, and therefore the ATP level controls the level of cAMP. Both Ca²⁺ and cAMP are very important second messengers. Ca²⁺ regulates almost every process in the human body (muscle contraction, blood coagulation, signal transfer in nerves, gene expression). Many enzymes require calcium ions as a cofactor, those of the blood-clotting cascade being notable examples. Extracellular calcium is also important for maintaining the potential difference across excitable cell membranes, as well as proper bone formation.

Mitochondria produce nitric oxide (NO) through a Ca²⁺-sensitive mitochondrial NO synthase (mtNOS). The NO produced by mtNOS regulates mitochondrial oxygen consumption and transmembrane potential via a reversible reaction with cytochrome c oxidase. The reaction of this NO with superoxide anion yields peroxynitrite, which irreversibly modifies susceptible targets within mitochondria and induces oxidative and/or nitrative stress. NO is an important cellular signaling molecule involved in many physiological and pathological processes. It is a powerful vasodilator with a short half-life of a few seconds in the blood. Low levels of nitric oxide production are important in protecting organs such as the liver from ischemic damage as well as paying a role on pain-regulating pathways.

The combination of the products of the reduction potential and reducing capacity of the linked redox couples present in cells and tissues represent the redox environment (redox state) of the cell. Redox couples present in the cell include: nicotinamide adenine dinucleotide (oxidized/reduced forms) NAD/NADH, nicotinamide adenine dinucleotide phosphate NADP/NADPH, glutathione/glutathione disulfide couple GSH/GSSG, and thioredoxin/thioredoxin disulfide couple Trx(SH)₂/TrxSS. Several important regulation pathways are mediated through the cellular redox state. Changes in redox state induce the activation of numerous intracellular signaling pathways, regulate nucleic acid synthesis, protein synthesis, enzyme activation and cell cycle progression. These cytosolic responses in turn induce transcriptional changes. Several transcription factors are regulated by changes in cellular redox state. Among them redox factor-1 (Ref-1)-dependent activator protein-1 (AP-1) (Fos and Jun), nuclear factor (B (NF-(B), p53, activating transcription factor/cAMP-response element-binding protein (ATF/CREB), hypoxia-inducible factor (HIF)-1alpha, an HIF-like factor.

Evaluating the efficiency of a biophotonic regimen at, for example, treating/healing skin (e.g., healing a wound) in real-time (i.e., during the course of a biophotonic regimen) may be achieved by assessing the activity of the cellular processes involved in cell survival and/or in cell death (e.g., programmed cell death, apoptosis). The biophotonic regimen of the present technology may, in some instances, promote cell survival pathways and/or may inhibit cell death pathways. Such may be assessed by determining the levels of one or more survival factors, growth factors, and/or death factors (such as, but not limited to: Bcl-xL, Cytochrome C, Caspase 9, Caspase 8, FADD, Bad, Bcl-2, Bax, PI3K, Akt, Akkα, IκB, NF-κB, PKC, PLC, or the like) that are involved in cell survival/cell death.

In view of the above disclosure and in view of the experimental data provided herein, one embodiment of the present technology is to provide a method for modulating a biophotonic treatment of a subject. In some implementations of this embodiment the method is performed during the course of a biophotonic regimen so as to obtain information in “real-time” about progression of the treatment and/or the healing. In some instances, the information in real-time indicates that the treatment/healing is progressing as expected. In some other instances, the information in real-time indicates that the treatment/healing is not progressing as expected (i.e., healing is slower than expected). In some other instances, the information in real-time indicates that treatment/healing is not occurring.

In some implementations of this embodiment, obtaining information about the progression of the treatment and/or healing is achieved by assaying the levels of energy production in the tissue undergoing biophotonic treatment (i.e., treated tissue) and assaying the levels of energy production in the tissue that is not undergoing biophotonic treatment (i.e., untreated tissue). In other implementations, obtaining information about the progression of the treatment and/or healing is achieved by assaying the levels of energy production in the tissue before undergoing biophotonic treatment (i.e., untreated tissue) and assaying the levels of energy production in the tissue after completion of the biophotonic treatment (i.e., treated tissue). In some instances, the method further comprises comparing the levels of energy production in the treated tissue versus the untreated. When the comparison shows that the levels of energy production in the treated tissue are higher than the levels of energy production in the untreated tissue it can be concluded that the parameters of the biophotonic regimen are efficient at treating/healing the tissue. Whereas when the comparison shows that the levels of energy production in the treated tissue are lower than the levels of energy production in the untreated tissue it can be concluded that the parameters of the biophotonic regimen are not efficient at treating/healing the tissue. In such instances, it is desirable to modulate the parameters of the biophotonic regimen so as to optimize the efficiency of the biophotonic regimen at treating/healing the tissue.

In one embodiment, the methods of the present technology, allow tailoring a biophotonic regimen to a specific tissue or to a specific subject so that the parameters of the biophotonic regimen are selected and chosen based on their efficiency at stimulating the treatment of the tissue or the subject. In some embodiments, the present technology relates to modulated/optimized biophotonic regimens that result from such tailoring.

In some embodiments, assaying cellular processes associated with production of energy is performed by assaying for the levels (e.g., concentration, expression, position, activity or the like) of cellular markers associated with energy production such as chemical compounds (molecules, proteins, enzymes, cofactors, sugars, etc.). Examples of cellular markers associated with energy production include, but are not limited to, chemical compounds involved in mitochondria function and/or mitochondrial biogenesis. Other examples of cellular markers involved in energy production include, but are not limited to, oxidative phosphorylation (OXPHOS), electron transport chain (ETC), and the citric acid cycle (CAC)/Kreb's cycle.

Further examples of cellular markers associated with energy production include, but are not limited to: ATP/ADP, ATP synthase, NADH/NAD, GTP/GDP, copy number of mitochondrial DNA, pyruvate, succinate, fumarate, co-enzyme A, pyruvate dehydrogenase, acetyl-CoA, citrate synthase, citrate, aconitase, isocitrate dehydrogenase, alpha-ketogluterate dehydrogenase, succinyl-CoA, succinyl CoC dehydrogenase, succinate dehydrogenase, fumarase, malate, malate dehydrogenase, oxalo acetate, citric acid, NADH-coenzyme Q oxidoreductase (complex I), succinate-Q oxidoreductase (complex II), electron transfer flavoprotein-Q oxidoreductase, Q-cytochrome c oxidoreductase (complex III), cytochrome c oxidase (complex IV). Methods for assaying for the levels of these cellular markers are well known in the art and may include techniques such as: protein and nucleotide isolation methods, gel electrophoresis, electrofocusing techniques, spectrometry, immunoassays, immunoprecipitation assays, crystallography, microscopy, protein footprinting, affinity purification, protein/nucleotide sequencing, proteomics, genomics, or the like.

Many methods are known in the art to measure mitochondrial function. Generally, measurements of fluxes give more information about the ability to make ATP than do measurements of intermediates and potentials. For isolated mitochondria, one assay is to measure the increase in respiration rate of the mitochondria in response to ADP. For intact cells, the best assay is the equivalent measurement of cell respiratory control, which reports the rate of ATP production, the proton leak rate, the coupling efficiency, the maximum respiratory rate, the respiratory control ratio and the spare respiratory capacity. Measurements of membrane potential provide useful additional information. Measurement of both respiration and potential during appropriate titrations enables the identification of the primary sites of effectors and the distribution of control, allowing deeper quantitative analyses. High-resolution respirometry has emerged as a powerful tool for in vitro measurements of mitochondrial function in isolated mitochondria and permeabilized fibers. Direct measurements of ATP production are possible by bioluminescence. Mechanistic data provided by these methods is further complimented by in vivo assessment using MRS and NIRS and the translational rate of gene transcripts.

Other techniques for assessing mitochondria function includes, but are not limited to: 1) assessing maximal ATP synthesis. The main function of mitochondria is to generate ATP by oxidizing nutrients (glucose, fatty acids, and some amino acids). In the tricarboxylic acid cycle (TCA), energy is released from acetyl groups as reduced coenzymes (NADH, FADH₂). Subsequently, the energy generated by electron transport is conserved by phosphorylation of ADP to ATP. The capacity for ATP synthesis is a property of tissue or mitochondria that is frequently used to define its functionality; 2) measuring maximal oxygen consumption. Oxygen is the final electron acceptor in the respiratory chain where it is reduced to water at complex IV (cytochrome c oxidase). Because the reduction of oxygen is a necessary precursor event to ATP synthesis, mitochondrial capacity is often assessed from the rates of oxygen consumption; 3) measuring mitochondrial coupling. Electron transport and ATP synthesis are tightly coupled, but some of the energy generated by electron transport is uncoupled from ATP synthesis. The efficiency of oxidative phosphorylation can be defined by the ratio of the number of moles of ATP generated for each atom of oxygen consumed (P/O ratio); and 4) measuring protein synthesis rates in vivo. Proper mitochondrial function is dictated to a large extent by the expression of mitochondrial proteins, but also by the integrity and functionality of individual mitochondrial proteins or protein complexes. Proteins undergo numerous posttranslational modifications that can interfere with their intended function such that increased expression of a particular protein may not necessarily reflect the abundance of functional proteins. Maintenance of a functional proteome is accomplished by the constant turnover of the protein pool as a consequence of degradation of old proteins and synthesis of new proteins to take their place. It is now possible to measure the synthesis rates of individual skeletal muscle mitochondrial proteins in vivo. Briefly, muscle proteins are labeled in vivo by intravenous infusion of L-[ring-¹³C₆]phenylalanine, followed by extraction and rapid freezing of tissues. Individual muscle proteins are purified by 2-dimensional gel electrophoresis, and the fractional synthesis rates of these proteins are calculated from the isotopic enrichment (measured by tandem mass spectrometry) of gel spots. This calculation is performed using the tissue fluid free phenylalanine enrichment as the precursor pool. This new methodology has wide-reaching applications since it can be performed in animals and in humans in combination with transcript levels of the specific proteins thus offering an opportunity to determine whether a condition or an intervention are accompanied by changes at the transcriptional or translational levels. Because the systemic infusion of isotope simultaneously labels all proteins being synthesized, it is possible to adapt this methodology to measure the synthesis rates of many proteins from nearly any organ tissue or biological fluid.

In some embodiments, the methods of the present technology comprise modulating and/or adjusting the parameters of the biophotonic regimen so as to optimize to the treatment/healing process. Adjusting the parameters of the biophotonic regimen may include for instance, changing and/or adjusting the wavelength at which the biophotonic regimen is carried out, changing and/or adjusting the power density of the light emitted during the illumination periods, changing and/or adjusting the concentration of the light-absorbing molecules or substituting the light-absorbing molecules for other types of light-absorbing molecules, adjusting the time of illumination, adjusting the recovery time between illumination periods, or the like.

According to various embodiments of the present technology, biophotonic regimens include application of a biophotonic composition onto the areas to be treated by phototherapy and illuminating the applied biophotonic composition for a period sufficient to activate the applied biophotonic composition.

Biophotonic compositions according to the present disclosure are compositions that are, in a broad sense, activated by light (e.g., photons) of a specific wavelength. These compositions comprises at least one light-absorbing molecule (e.g., endogenous or exogenous), which is activated by light and accelerates the dispersion of light energy, which leads to light carrying on a therapeutic effect on its own, and/or to the photochemical activation of other agents contained in the composition.

The compositions of the present disclosure are activated by light (e.g., photons) of specific wavelength. The compositions comprise at least one light-absorbing molecule which is activated by light and accelerates the dispersion of light energy, which leads to light carrying on a therapeutic effect on its own, and/or to the photochemical activation of other agents present in the composition.

When a light-activating molecule absorbs a photon of a certain wavelength, it becomes excited. This is an unstable condition and the light-activating molecule tries to return to the ground state, giving away the excess energy. For some light-activating molecules, it is favorable to emit the excess energy as light when transforming back to the ground state. This process is called fluorescence. The peak wavelength of the emitted fluorescence is shifted towards longer wavelengths compared to the absorption wavelengths (i.e., Stokes' shift). The emitted fluorescent energy can then be transferred to the other components of the composition or to a treatment site on to which the composition is topically applied. Differing wavelengths of light may have different and complementary therapeutic effects on tissue.

In certain implementations, the compositions of the present disclosure are substantially transparent. In certain embodiments, the compositions of the present disclosure are substantially translucent. In some certain embodiments, the compositions of the present disclosure have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths. The % transmittance of the composition can be measured in the range of wavelengths from 250 nm to 800 nm using, for example, a Perkin-Elmer Lambda 9500 series UV-visible spectrophotometer. Alternatively, a Synergy HT spectrophotometer (BioTek Instrument, Inc.) can be used in the range of wavelengths from 380 nm to 900 nm. Transmittance is calculated according to the following equation:

$${A}_{λ} = {log}_{10} \frac{{I}_{0}}{I} = {log}_{10} \frac{1}{T} .$$

where A is absorbance, T is transmittance, I₀ is intensity of radiation before passing through material, and I is intensity of light passing through material.

The values can be normalized for thickness. As stated herein, % transmittance (translucency) is as measured for a 2 mm thick sample at a wavelength of 526 nm. It will be clear that other wavelengths, thickness of the composition or the like can be used.

In some instances, the compositions of the present disclosure are for topical uses (i.e., suitable for topical application). The composition can be in the form of a solid, semi-solid or viscous liquid, such as a gel, or are gel-like, and which have a spreadable consistency at room temperature (e.g., about 20-25° C.) prior to illumination. In certain such instances wherein the composition has a spreadable consistency, the composition can be topically applied to a treatment site at a thickness of from about 0.5 mm to about 3 mm, from about 0.5 mm to about 2.5 mm, or from about 1 mm to about 2 mm. The composition can be topically applied to a treatment site at a thickness of about 2 mm or about 1 mm Spreadable compositions can conform to a topography of an application site. This can have advantages over a non-conforming material in that a better and/or more complete illumination of the application site can be achieved and the compositions are easy to apply and remove.

In some embodiments, the composition has a transparency or translucency that exceeds 1%, 2%, 3%, 4%, 5%, 10%, 15%, 20%, 25%, 30%, 35%, 40%, 45%, 50%, 55%, 60%, 65%, 70%, 75%, 80%, or 85%. In some embodiments, the transparency exceeds 70%, 80%, 85%, 86%, 87%, 88%, 89%, 90%, 91%, 92%, 93%, 94%, 95%, 96%, 97%, 98% or 99%. All transmittance values reported herein are as measured on a 2 mm thick sample using the Synergy HT spectrophotometer at a wavelength of 526 nm.

In some aspects, the compositions of the present disclosure comprise at least a first light-absorbing molecule in a medium, wherein the composition is substantially resistant to leaching such that a low or negligible amount of the light-absorbing molecule leaches out of the composition into for example skin or onto a soft tissue onto which the composition is applied. In certain embodiments, this is achieved by the medium comprising a gelling agent which slows or restricts movement or leaching of the light-absorbing molecule.

Suitable light-absorbing molecules can be fluorescent dyes (or stains), although other dye groups or dyes (biological and histological dyes, food colorings, carotenoids, and other dyes) can also be used. Suitable light-absorbing molecules can be those that are Generally Regarded As Safe (GRAS), although light-absorbing molecules which are not well tolerated by the skin or other tissues can be included in the composition as contact with the skin is minimal in use due to the leaching-resistant nature of the composition.

Other suitable light-absorbing molecules can be endogenous light-absorbing molecules such as, but not limited to, vitamins. Examples of vitamins that may act as endogenous light-absorbing molecules include, vitamin B. In some instances, the endogenous light-absorbing molecule is vitamin B12. In some instances, the endogenous light-absorbing molecule is 7,8-Di methyl-10-[(2S,3S,4R)-2,3,4,5-tetrahydroxypentyl]benzo[g]pteridine-2,4-dione.

In certain embodiments, the composition of the present disclosure comprises at least one light-absorbing molecule which undergoes partial or complete photobleaching upon application of light. In some embodiments, the at least one light-absorbing molecule absorbs and/or emits at a wavelength in the range of the visible spectrum, such as at a wavelength of between about 380 nm and about 1 mm, between 380 nm and 800 nm, between about 380 nm and about 700 nm, or between about 380 nm and about 600 nm. In other embodiments, the at least one light-absorbing molecule absorbs/or emits at a wavelength of between about 200 nm and about 800 nm, between about 200 nm and about 700 nm, between about 200 nm and about 600 nm or between about 200 nm and about 500 nm. In other embodiments, the at least one light-absorbing molecule absorbs/or emits at a wavelength of between about 200 nm and about 1 mm In other embodiments, the at least one light-absorbing molecule absorbs/or emits at a wavelength of between about 700 nm and about 1 mm In other embodiments, the at least one light-absorbing molecule absorbs/or emits at a wavelength of between about 200 nm and about 600 nm. In some embodiments, the at least one light-absorbing molecule absorbs/or emits light at a wavelength of between about 200 nm and about 300 nm, between about 250 nm and about 350 nm, between about 300 nm and about 400 nm, between about 350 nm and about 450 nm, between about 400 nm and about 500 nm, between about 400 nm and about 600 nm, between about 450 nm and about 650 nm, between about 600 nm and about 700 nm, between about 650 nm and about 750 nm or between about 700 nm and about 800 nm.

It will be appreciated to those skilled in the art that optical properties of a particular light-absorbing molecule may vary depending on the light-absorbing molecule's surrounding medium. Therefore, as used herein, a particular light-absorbing molecule's absorption and/or emission wavelength (or spectrum) corresponds to the wavelengths (or spectrum) measured in a composition useful in the methods of the present disclosure.

In some instances, the light-absorbing molecule of the composition is selected from a xanthene derivative dye, an azo dye, a biological stain, and a carotenoid. In some instances, the at least one light-absorbing molecule is selected from eosin (e.g., eosin B or eosin Y), erythrosine (e.g., erythrosine B), fluorescein, Rose Bengal, and Saffron red powder.

In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin 0, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, alpha-carotene, beta-carotene, bixin, and fucoxanthine. In certain such embodiments, said carotenoid is present in the composition as a mixture is selected from saffron red powder, annatto extract, and brown algae extract.

In some embodiments, the at least one light-absorbing molecule is present in an amount of between about 0.001% and 40% by weight of the composition. In some embodiments, the at least one light-absorbing molecule is present in an amount of between about 0.005% and about 2%, between about 0.01% and about 1%, between about 0.01% and about 2%, between about 0.05% and about 1%, between about 0.05% and about 2%, between about 0.1% and about 1%, between about 0.1% and about 2%, between about 1% and about 5%, about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% by weight of the composition. In some embodiments, the at least one light-absorbing molecule is present in an amount of at least about 0.2% by weight of the composition.

The compositions disclosed herein may include at least one additional light-absorbing molecule. Combining light-absorbing molecules may increase photo-absorption by the combined dye molecules and enhance absorption and photo-biomodulation selectivity. This creates multiple possibilities of generating new photosensitive, and/or selective light-absorbing molecule mixtures.

When such multi-light-absorbing molecule compositions are illuminated with light, energy transfer can occur between the light-absorbing molecules. This process, known as resonance energy transfer, is a photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule). The efficiency and directedness of resonance energy transfer depends on the spectral features of donor and acceptor light-absorbing molecule. In particular, the flow of energy between light-absorbing molecules is dependent on a spectral overlap reflecting the relative positioning and shapes of the absorption and emission spectra. For energy transfer to occur the emission spectrum of the donor light-absorbing molecule overlap with the absorption spectrum of the acceptor light-absorbing molecule. Energy transfer manifests itself through decrease or quenching of the donor emission and a reduction of excited state lifetime accompanied also by an increase in acceptor emission intensity. To enhance the energy transfer efficiency, the donor light-absorbing molecule should have good abilities to absorb photons and emit photons. Furthermore, it is thought that the more overlap there is between the donor light-absorbing molecule's emission spectra and the acceptor light-absorbing molecule's absorption spectra, the better a donor light-absorbing molecule can transfer energy to the acceptor light-absorbing molecule.

In some embodiments, the donor, or first, light-absorbing molecule has an emission spectrum that overlaps at least about 80%, about 70%, about 60%, about 50%, about 40%, about 30%, about 20%, or about 10%, or about 5%, or about 2%, or about 1% with an absorption spectrum of the second light-absorbing molecule. In some embodiments, the first light-absorbing molecule has an emission spectrum that overlaps at least about 20% with an absorption spectrum of the second light-absorbing molecule. In some embodiments, the first light-absorbing molecule has an emission spectrum that overlaps at least between about 1% and about 10%, between about 5% and about 15%, between about 10% and about 20%, between about 15% and about 25%, between about 20% and about 30%, between about 25% and about 35%, between about 30% and about 40%, between about 35% and about 45%, between about 50% and about 60%, between about 55% and about 65% or between about 60% and about 70% with an absorption spectrum of the second light-absorbing molecule. Percent (%) spectral overlap, as used herein, refers to the % overlap of a donor light-absorbing molecule's emission wavelength range with an acceptor light-absorbing molecule's absorption wavelength range, measured at spectral full width quarter maximum (FWQM). In some embodiments, the second light-absorbing molecule absorbs at a wavelength in the range of the visible spectrum. In some embodiments, the second light-absorbing molecule has an absorption wavelength that is relatively longer than that of the first light-absorbing molecule within the range of between about 50 nm and about 250 nm, between about 25 nm and about 150 nm or between about 10 nm and about 100 nm.

As discussed above, the application of light to the compositions of the present disclosure can result in a cascade of energy transfer between the light-absorbing molecules. In some embodiments, such a cascade of energy transfer provides photons that penetrate the epidermis, dermis and/or mucosa (or even lower) at the target tissue.

In some embodiments, the light-absorbing molecule is selected such that their emitted fluorescent light, on photoactivation, is within one or more of the green, yellow, orange, red and infrared portions of the electromagnetic spectrum, for example having a peak wavelength within the range of about 490 nm to about 1 mm In some embodiments, the emitted fluorescent light has a power density of between 0.005 mW/cm² to about 10 mW/cm², about 0.5 mW/cm² to about 5 mW/cm².

Further examples of suitable light-absorbing molecules useful in the compositions, methods, and uses of the present disclosure include, but are not limited to the following: Xanthene derivatives—The xanthene group comprises three sub-groups: a) the fluorenes; b) fluorones; and c) the rhodoles, any of which may be suitable for the compositions, methods, and uses of the present disclosure. The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group. The fluorone group comprises the fluorescein dye and the fluorescein derivatives. Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds. The eosins group comprises Eosin Y (tetrabromofluorescein, acid red 87, D&C Red 22), a light-absorbing molecule with an absorption maximum of 514-518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin”, and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present is an amount of at about 0.005% by weight of the total composition. In some embodiments, at least one of Eosin B or Eosin Y or a combination thereof is present is an amount of at about 0.01% by weight of the total composition. In some embodiments, at least one of Eosin B or Eosin Y or a combination thereof is present is an amount of at about 0.02% by weight of the total composition. In some embodiments, at least one of Eosin B or Eosin Y or a combination thereof is present is an amount of at about 0.05% by weight of the total composition. In some embodiments, at least one of Eosin B or Eosin Y or a combination thereof is present is an amount of at about 0.1% by weight of the total composition. In some embodiments, at least one of Eosin B or Eosin Y or a combination thereof is present is an amount of at about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition. In some embodiments, the composition includes in the range of less than 12% by weight of the total composition of at least one of Eosin B or Eosin Y or a combination thereof. In some embodiments, at least one of Eosin B or Eosin Y or a combination thereof is present from 0.001% to 12%, or between 0.01% and 1.2%, or from 0.01% to 0.5%, or from 0.1% to 0.5%, or from 0.5% to 0.8%, or from 0.01% to 0.05%, by weight of the total composition. In some embodiments, at least one of Eosin B or Eosin Y or a combination thereof is present is an amount of at 0.005% by weight of the total composition. In some embodiments, at least one of Eosin B or Eosin Y or a combination thereof is present is an amount of at 0.01% by weight of the total composition. In some embodiments, at least one of Eosin B or Eosin Y or a combination thereof is present is an amount of at 0.02% by weight of the total composition. In some embodiments, at least one of Eosin B or Eosin Y or a combination thereof is present is an amount of at 0.05% by weight of the total composition. In some embodiments, at least one of Eosin B or Eosin Y or a combination thereof is present is an amount of at 0.1% by weight of the total composition. In some embodiments, at least one of Eosin B or Eosin Y or a combination thereof is present is an amount of at 0.2% by weight of the total composition. In some embodiments, at least one of Eosin B or Eosin Y or a combination thereof is present is an amount of at least 0.2% by weight of the total composition but less than 1.2% by weight of the total composition. In some embodiments, at least one of Eosin B or Eosin Y or a combination thereof is present is an amount of at least 0.01% by weight of the total composition but less than 12% by weight of the total composition. Phloxine B (2,4,5,7 tetrabromo 4,5,6,7,tetrachlorofluorescein, D&C Red 28, acid red 92) is a red dye derivative of fluorescein which is used for disinfection and detoxification of waste water through photooxidation. It has an absorption maximum of 535-548 nm. It is also used as an intermediate for making photosensitive dyes and drugs. Erythrosine B, or simply Erythrosine or Erythrosin (acid red 51, tetraiodofluorescein) is a cherry-pink, coal-based fluorine food dye used as a biological stain, and a biofilm and dental plaque disclosing agent, with a maximum absorbance of 524-530 nm in aqueous solution. It is subject to photodegradation. In embodiments, the composition includes in the range of less than about 2% by weight Erythrosine B. In some embodiments, Erythrosine B is present in an amount from about 0.005 to about 2%, or from about 0.005% to about 1%, or about 0.01% to about 1% by weight of the total composition. In some embodiments, Erythrosine B is present in an amount of about 0.005% and about 0.15% by weight of the total composition. Rose Bengal (4,5,6,7 tetrachloro 2,4,5,7 tetraiodofluorescein, acid red 94) is a bright bluish-pink fluorescein derivative with an absorption maximum of 544-549 nm, that has been used as a dye, biological stain and diagnostic aid. Merbromine (mercurochrome) is an organo-mercuric disodium salt of fluorescein with an absorption maximum of 508 nm. It is used as an antiseptic. Azo dyes—The azo (or diazo-) dyes share the N—N group, called azo the group. They are used mainly in analytical chemistry or as food colorings and are not fluorescent. Suitable azo dyes for the compositions, methods, and uses of the disclosure include: Methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate. Biological stains—Suitable biological stains include: Safranin (Safranin 0, basic red 2) is an azo-dye and is used in histology and cytology. Fuchsin (basic or acid) (rosaniline hydrochloride) is a magenta biological dye that can stain bacteria and has been used as an antiseptic. 3,3′ dihexylocarbocyanine iodide (DiOC6) is a fluorescent dye used for staining the endoplasmic reticulum, vesicle membranes and mitochondria of cells. It shows photodynamic toxicity; when exposed to blue light, has a green fluorescence. Carminic acid (acid red 4, natural red 4) is a red glucosidal hydroxyanthrapurin naturally obtained from cochineal insects. Indocyanin green (ICG) is used as a diagnostic aid for blood volume determination, cardiac output, or hepatic function. ICG binds strongly to red blood cells and when used in mixture with fluorescein, it increases the absorption of blue to green light. Carotenoids—Carotenoid dyes are also photoactivators that are useful in the compositions, methods, and uses of the disclosure. Saffron red powder is a natural carotenoid-containing compound. Saffron is a spice derived from Crocus sativus. It is characterized by a bitter taste and iodoform or hay-like fragrance; these are caused by the compounds picrocrocin and saffranal. It also contains the carotenoid dye crocin that gives its characteristic yellow-red color. Saffron contains more than 150 different compounds, many of which are carotenoids: mangicrocin, reaxanthine, lycopene, and various carotenes, which show good absorption of light and beneficial biological activity. Also saffron can act as both a photon-transfer agent and a healing factor. Saffron color is primarily the result of a-crocin (8,8 diapo-8,8-carotenoid acid). Dry saffron red powder is highly sensitive to fluctuating pH levels and rapidly breaks down chemically in the presence of light and oxidizing agents. It has a deep red colour and forms crystals with a melting point of 186° C. Crocetin, another compound of saffron, was found to express an antilipidemic action and promote oxygen penetration in different tissues. Fucoxanthine is a constituent of brown algae with a pronounced ability for photosensitization of redox reactions. Chlorophyll dyes—Examples of chlorophyll dyes that are useful in the compositions, methods, and uses of the disclosure, include but are not limited to chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2.

In some aspects of the disclosure, the one or more light-absorbing molecules of the composition disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Safranin 0), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments, the composition includes Eosin Y as a first light-absorbing molecule. In some embodiments, the composition includes Eosin Y as a first light-absorbing molecule and any one or more of Rose Bengal, Fluorescein, Erythrosin, Phloxine B as a second light-absorbing molecule.

In some embodiments, the composition includes the following synergistic combinations: Eosin Y and Fluorescein; Fluorescein and Rose Bengal; Erythrosine in combination with one or more of Eosin Y, Rose Bengal or Fluorescein; or Phloxine B in combination with one or more of Eosin Y, Rose Bengal, Fluorescein and Erythrosine. Other synergistic light-absorbing molecule combinations are also possible.

By means of synergistic effects of the light-absorbing molecule combinations in the composition, light-absorbing molecules which cannot normally be activated by an activating light (such as a blue light from an LED) can be activated through energy transfer from the light-absorbing molecules which are activated by the activating light. In this way, the different properties of photoactivated light-absorbing molecules can be harnessed and tailored according to the therapy required.

Light-absorbing molecule combinations can also have a synergistic effect in terms of their photoactivated state. For example, two light-absorbing molecules may be used, one of which emits fluorescent light when activated in the blue and green range, and the other which emits fluorescent light in the red, orange and yellow range, thereby complementing each other and irradiating the target tissue with a broad wavelength of light having different depths of penetration into target tissue and different therapeutic effects.

In some embodiments, the present disclosure provides compositions that comprise at least a first light-absorbing molecule and a gelling agent. A gelling agent may comprise any ingredient suitable for use in a topical composition as described herein. The gelling agent may be an agent capable of forming a cross-linked matrix, including physical and/or chemical cross-links The gelling agent is preferably biocompatible, and may be biodegradable. In some implementations, the gelling agent is able to form a hydrogel or a hydrocolloid. An appropriate gelling agent is one that can form a viscous liquid or a semisolid. In preferred embodiments, the gelling agent and/or the composition has an appropriate light transmission property. The gelling agent preferably allows activity of the light-absorbing molecule(s). For example, some light-absorbing molecules require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines.

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity.

Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6. In some implementations, the carbomer is Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. Carbopols are versatile controlled-release polymers and belong to a family of carbomers which are synthetic, high molecular weight, non-linear polymers of acrylic acid, crosslinked with polyalkenyl polyether. In some embodiments, the carbomer is Carbopol® 974P NF, 980 NF, 5984 EP, ETD 2020NF, Ultrez 10 NF, 934 NF, 934P NF or 940 NF. In some embodiments, the carbomer is Carbopol® 980 NF, ETD 2020 NF, Ultrez 10 NF, Ultrez 21 or 1382 Polymer, 1342 NF, 940 NF. In some embodiments, from about 0.05% to about 10%, about 0.5% to about 5%, or about 1% to about 3% by weight of the total composition of a high molecular weight carbopol can be present as the gelling agent. In some embodiments, the biophotonic composition of the disclosure comprises from about 0.05% to about 10%, about 0.5% to about 5%, or from about 1% to about 3% by weight of the total composition of a high molecular weight carbopol.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, poly(vinyl alcohol), poly(2-hydroxyethylmethylacrylate), polyethylene oxide, collagen, chitosan, alginate, a poly(acrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the composition can include up to about 2% by weight of the final composition of sodium hyaluronate as the single gelling agent. In some embodiments, the composition can include more than about 4% or more than about 5% by weight of the total composition of gelatin as the single gelling agent. In some embodiments, the composition can include up to about 10% or up to about 8% starch as the single gelling agent. In some embodiments, the composition can include more than about 5% or more than about 10% by weight of the total composition of collagen as the gelling agent. In some embodiments, about 0.1% to about 10% or about 0.5% to about 3% by weight of the total composition of chitin can be used as the gelling agent. In some embodiments, about 0.5% to about 5% by weight of the final composition of corn starch or about 5% to about 10% by weight of the total composition of corn starch can be used as the gelling agent. In some embodiments, more than about 2.5% by weight of the total composition of alginate can be used in the composition as the gelling agent. In some embodiments, the percentages by weight percent of the final composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylypyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

In some embodiments, the compositions of the present disclosure further comprise oxidants such as for instance, peroxide compounds are that contain the peroxy group (R—O—O—R). In some embodiments, the biophotonic compositions of this disclosure comprises an oxidant selected from, but not limited to, hydrogen peroxide, carbamide peroxide, benzoyl peroxide, peroxy acids, or alkali metal percarbonates

In the compositions and methods of the present disclosure, additional components may optionally be included, or used in combination with the compositions as described herein. Such additional components include, but are not limited to, chelating agents, polyols, healing factors, growth factors, antimicrobials, wrinkle fillers (e.g. botox, hyaluronic acid or polylactic acid), collagens, anti-virals, anti-fungals, antibiotics, drugs, and/or agents that promote collagen synthesis. These additional components may be applied to the wound, skin or mucosa in a topical fashion, prior to, at the same time of, and/or after topical application of the composition of the present disclosure, and may also be systemically administered. Suitable healing factors, antimicrobials, collagens, and/or agents that promote collagen synthesis are discussed below:

Healing factors comprise compounds that promote or enhance the healing or regenerative process of the tissues on the application site of the composition. During the photoactivation of the composition of the present disclosure, there may be an increase of the absorption of molecules at the treatment site by the skin, wound or the mucosa. An augmentation in the blood flow at the site of treatment is observed for a period of time. An increase in the lymphatic drainage and a possible change in the osmotic equilibrium due to the dynamic interaction of the free radical cascades can be enhanced or even fortified with the inclusion of healing factors. Suitable healing factors include, but are not limited to: hyaluronic acid, glucosamine, allantoin, saffron.

Examples of antimicrobials (or antimicrobial agent) are recited in U.S. Patent Application Publication Nos: 2004/0009227 and 2011/0081530, which are both herein incorporated by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

In some embodiments, the pH of the composition is in or adjusted to the range of about 4 to about 10. In some embodiments, the pH of the composition is in or adjusted to the range of about 4 to about 9. In some embodiments, the pH of the composition is in or adjusted to the range of about 4 to about 8. In some embodiments, the pH of the composition is within the range of about 4 to about 7. In some embodiments, the pH of the composition is within the range of about 4 to about 6.5. In some embodiments, the pH of the composition is within the range of about 4 to about 6. In some embodiments, the pH of the composition is within the range of about 4 to about 5.5. In some embodiments, the pH of the composition is within the range of about 4 to about 5. In some embodiments, the pH of the composition is within the range of about 5.0 to about 8.0. In some embodiments, the pH of the composition is within the range of about 6.0 to about 8.0. In some embodiments, the pH of the composition is within the range of about 6.5 to about 7.5. In some embodiments, the pH of the composition is within the range of about 5.5 to about 7.5.

In some embodiments, the pH of the composition is in or adjusted to the range of 4 to 10. In some embodiments, the pH of the composition is in or adjusted to the range of 4 to 9. In some embodiments, the pH of the composition is in or adjusted to the range of 4 to 8. In some embodiments, the pH of the composition is within the range of 4 to 7. In some embodiments, the pH of the composition is within the range of 4 to 6.5. In some embodiments, the pH of the composition is within the range of 4 to 6. In some embodiments, the pH of the composition is within the range of 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5.

In some embodiments, the compositions of the disclosure also include an aqueous substance (water) or an alcohol. Alcohols include, but are not limited to, ethanol, propanol, isopropanol, butanol, iso-butanol, t-butanol or pentanol. In some embodiments, the light-absorbing molecule or combination of light-absorbing molecules is in solution in a medium of the composition. In some embodiments, the light-absorbing molecule or combination of light-absorbing molecules is in solution in a medium of the composition, wherein the medium is an aqueous substance.

In some implementations of the embodiments of the present disclosure, the biophotonic compositions of the present disclosure may promote wound healing or tissue repair, especially in non-healing wounds. The biophotonic compositions of the present disclosure may also be used for treating acute inflammation, especially in non-healing wounds. Therefore, in some aspects, the present disclosure may provide for a method of providing biophotonic therapy to a non-healing wound, where the method promotes or stimulates healing of that wound.

In some embodiments, the methods of the present disclosure comprise applying a composition of the present disclosure to an area of the skin of a subject that is in need of phototherapy and illuminating the applied composition with light having a wavelength that overlaps with an absorption spectrum of the at least one light-absorbing molecule of the composition. In some implementations, the composition is applied topically.

In the methods of the present disclosure, any source of actinic light can be used to illuminate the compositions. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some instances, an argon laser is used. In some instances, a potassium-titanyl phosphate (KTP) laser (e.g., a GreenLight™ laser) is used. In other instances, sunlight may be used. In some instances, a LED photocuring device is the source of the actinic light. The source of the actinic light is a source of light having a wavelength between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, between about 380 nm and about 550 nm, between about 200 nm and about 800 nm, between about 400 nm and about 700 nm, between about 400 nm and about 600 nm, between about 400 nm and about 550 nm, between about 380 nm and about 700 nm, between about 380 nm and about 600 nm, or between about 380 nm and about 550 nm. In some instances, the composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 1200 mW/cm², such as from about 20 mW/cm² to about 1000 mW/cm² from about 100 mW/cm² to about 900 mW/cm² from about 200 mW/cm² to about 800 mW/cm², or from about 1 mW/cm² to about 200 mW/cm². In some embodiments, the power density for non-collimated light sources (LED, halogen or plasma lamps) are in the range from about 1 mW/cm² to about 200 mW/cm² Suitable power density for laser light sources is in the range from about 0.5 mW/cm² to about 0.8 mW/cm².

In some embodiments of the methods of the present disclosure, the light has an energy at the subject's skin of from about 1 mW/cm² to about 500 mW/cm², or about 1 mW/cm² to about 300 mW/cm², or about 1 mW/cm² to about 200 mW/cm², wherein the energy applied depends at least on the condition being treated, the wavelength of the light, the distance of the subject's skin from the light source, and the thickness of the composition. In some embodiments, the light at the subject's skin is from about 1 mW/cm² to about 40 mW/cm², or about 20 mW/cm² to about 60 mW/cm², or about 40 mW/cm² to about 80 mW/cm², or about 60 mW/cm² to about 100 mW/cm², or about 80 mW/cm² to about 120 mW/cm², or about 100 mW/cm² to about 140 mW/cm², or about 120 mW/cm² to about 160 mW/cm², or about 140 mW/cm² to about 180 mW/cm², or about 160 mW/cm² to about 200 mW/cm², or about 110 mW/cm² to about 240 mW/cm², or about 110 mW/cm² to about 150 mW/cm², or about 190 mW/cm² to about 240 mW/cm².

In some embodiments, the light-activating molecule can be photoactivated by ambient light which may originate from the sun or other light sources. Ambient light can be considered to be a general illumination that comes from all directions in a room that has no visible source. The light-activating molecule can be photoactivated by light in the visible range of the electromagnetic spectrum. Exposure times to ambient light may be longer than that to direct light.

In some embodiments, different sources of light can be used to activate the compositions, such as a combination of ambient light and direct LED light. The duration of the exposure to actinic light required will be dependent on the surface of the treated area, the severity of the condition that is being treated, the power density, wavelength and bandwidth of the light source, the thickness of the composition, and the treatment distance from the light source. The illumination of the treated area by fluorescence may take place within seconds or even fragment of seconds, but a prolonged exposure period is beneficial to exploit the synergistic effects of the absorbed, reflected and reemitted light on the composition of the present disclosure and its interaction with the tissue being treated. In some embodiments, the time of exposure to actinic light of the tissue or skin which the composition has been applied is a period from about 1 second to about 30 minutes, from about 1 minute to about 30 minutes, from about 1 minute to about 5 minutes, from about 1 minute to about 5 minutes, from about 20 seconds to about 5 minutes, from about 60 seconds to about 5 minutes, or for less than about 5 minutes, or between about 20 seconds to about 5 minutes, or from about about 60 seconds to about 5 minutes per cm² of the area to be treated, so that the total time of exposure of a 10 cm² area would be from about 10 minutes to about 50 minutes.

In certain embodiments, the fluence delivered to the treatment areas may be between about 1 to about 60 J/cm², about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm².

In some embodiments, the composition is illuminated for a period from about 1 minute and 3 minutes. In some embodiments, light is applied for a period of from about 1 second to about 30 seconds, from about 1 second to about 60 seconds, from about 15 seconds to about 45 seconds, from about 30 seconds to about 60 seconds, from about 0.75 minute to about 1.5 minutes, from about 1 minute to about 2 minutes, from about 1.5 minutes to about 2.5 minutes, from about 2 minutes to about 3 minutes, from about 2.5 minutes to about 3.5 minutes, from about 3 minutes to about 4 minutes, from about 3.5 minutes to about 4.5 minutes, from about 4 minutes to about 5 minutes, from about 5 minutes to about 10 minutes, from about 10 minutes to about 15 minutes, from about 15 minutes to about 20 minutes, from about 20 minutes to about 25 minutes, or from about 20 minutes to about 30 minutes. In some embodiments, light is applied for a period of 1 second, about 5 seconds, about 10 seconds, about 20 seconds, about 30 seconds, less than about 30 minutes, less than about 20 minutes, less than about 15 minutes, less than about 10 minutes, less than about 5 minutes, less than about 1 minute, less than about 30 seconds, less than about 20 seconds, less than 10 seconds, less than 5 seconds, or for less than 1 second.

In some embodiments, the source of actinic light is in continuous motion over the treated area for the appropriate time of exposure. In some instances, multiple applications of the composition and actinic light are performed. In some instances, the tissue or skin is exposed to actinic light at least two, three, four, five or six times. In some embodiments, the tissue or skin is exposed to actinic light at least two, three, four, five or six times with a resting period in between each exposure. In certain such embodiments, the resting period is less than about 1 minute, less than about 5 minutes, less than about 10 minutes, less than about 20 minutes, less about 40 minutes, less than about 60 minutes, less than about 2 hours, less than about 4 hours, less than about 6 hours, or less than 12 hours. In some embodiments, the entire treatment may be repeated in its entirety as may be required by the patient. In some embodiments, a fresh application of the composition is applied before another exposure to actinic light.

In the methods of the present disclosure, the composition may be optionally removed from the site of treatment following application of light. In some instances, the composition is left on the treatment site for more than about 30 minutes, more than one hour, more than about 2 hours, or more than about 3 hours. It can be illuminated with ambient light. To prevent drying, the composition can be covered with a transparent or translucent cover such as a polymer film, or an opaque cover which can be removed before illumination.

The compositions of the disclosure may be applied at regular intervals such as once a week. The compositions of the disclosure may be applied once per week for one or more weeks, such as once per week for one week. The compositions of the disclosure may be applied once per week for two weeks, once per week for three weeks, once per week for four weeks, once per week for five weeks, once per week for six weeks, once per week for seven weeks, or once per week for eight or more weeks.

The compositions of the disclosure may be applied twice per week for one or more weeks, such as twice per week for one week. The compositions of the disclosure may be applied twice per week for two weeks, twice per week for three weeks, twice per week for four weeks, twice per week for five weeks, twice per week for six weeks, twice per week for seven weeks, or twice per week for eight or more weeks. The compositions of the disclosure may be applied three times or more per week for one or more weeks, such as three times or more for one week. The compositions of the disclosure may be applied three times or more per week for two weeks, three times or more per week for three weeks, three times or more per week for four weeks, three times or more per week for five weeks, three times or more per week for six weeks, three times or more per week for seven weeks, or three times or more per week for eight or more weeks.

The biophotonic compositions and methods of the present disclosure may be used to treat wounds. Examples of wounds that may be treated by the present technology include, for example, skin diseases that result in a break of the skin or in a wound, clinically infected wounds, burns, incisions, excisions, lesions, lacerations, abrasions, puncture or penetrating wounds, gunshot wounds, surgical wounds, contusions, hematomas, crushing injuries, ulcers, scarring (cosmesis), wounds caused by periodontitis. The biophotonic compositions and methods of the present disclosure may be used to treat acute wounds. The biophotonic compositions and methods of the present disclosure may be used to treat chronic wounds. Chronic wound means a wound that has not healed within about 4 to 6 weeks. Chronic wounds include venous ulcers, venous stasis ulcers, arterial ulcers, pressure ulcers, diabeteic ulcers, ulcers due to arterial insufficiency and diabetic foot ulcers. The biophotonic compositions and methods of the present disclosure may be used for cosmesis.

The biophotonic compositions and methods of the present disclosure may be used to treat wounds The biophotonic compositions and methods of the present disclosure may be used to treat non-healing wounds and promote healing or granulation tissue formation. Non-healing wounds that may be treated by the biophotonic compositions and methods of the present disclosure include, for example, those arising from acute wounds, injuries to the skin and subcutaneous tissue initiated in different ways (e.g., pressure ulcers from extended bed rest or from being in a non-ambulatory state or due to a presence (whether repeated or chronic) of an external factor such as a therapeutic device such as a cast or a non-therapeutic device such as a saddle or similar device for a non-human animal), wounds induced by trauma, wounds induced by conditions such as periodontitis, wounds induced by inflammation, wounds induced by infection, or the like), and with varying characteristics. In certain embodiments, the present disclosure provides biophotonic compositions and methods for treating and/or promoting the healing of, for example, skin diseases that result in a break of the skin or in a wound, clinically infected wounds, burns, incisions, excisions, lacerations, abrasions, puncture or penetrating wounds, gun-shot wounds, surgical wounds, contusions, hematomas, crushing injuries, sores and ulcers.

Biophotonic compositions and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

In certain other embodiments, the present disclosure provides biophotonic compositions and methods for treating and/or promoting healing, Grade I-IV ulcers. In certain embodiments, the application provides compositions suitable for use with Grade II and Grade III ulcers in particular. Ulcers may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

For example, the present disclosure provides biophotonic compositions and methods for treating and/or promoting healing of a diabetic ulcer. Diabetic patients are prone to foot and other ulcerations due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. A patient with advanced neuropathy loses the ability to sense a sustained pressure insult, as a result, tissue ischemia and necrosis may occur leading to for example, plantar ulcerations. Microvascular disease is one of the significant complications for diabetics which may also lead to ulcerations. In certain embodiments, compositions and methods of treating a chronic wound are provided here in, where the chronic wound is characterized by diabetic foot ulcers and/or ulcerations due to neurologic and/or vascular complications of diabetes.

In other examples, the present disclosure provides biophotonic compositions and methods for treating and/or promoting healing of a pressure ulcer. Pressure ulcer includes bed sores, decubitus ulcers and ischial tuberosity ulcers and can cause considerable pain and discomfort to a patient. A pressure ulcer can occur as a result of a prolonged pressure applied to the skin. Thus, pressure can be exerted on the skin of a patient due to the weight or mass of an individual. A pressure ulcer can develop when blood supply to an area of the skin is obstructed or cut off for more than two or three hours. The affected skin area can turns red, becomes painful and can become necrotic. If untreated, the skin breaks open and can become infected. An ulcer sore is therefore a skin ulcer that occurs in an area of the skin that is under pressure from e.g. lying in bed, sitting in a wheelchair, and/or wearing a cast for a prolonged period of time. Pressure ulcer can occur when a person is bedridden, unconscious, unable to sense pain, or immobile. Pressure ulcer often occur in honey prominences of the body such as the buttocks area (on the sacrum or iliac crest), or on the heels of foot.

Wound healing in adult tissues is a complicated reparative process. For example, the healing process for skin involves the recruitment of a variety of specialized cells to the site of the wound, extracellular matrix and basement membrane deposition, angiogenesis, selective protease activity and re-epithelialization. There are four overlapping phases in the normal wound healing process. First, in the hemostasis and inflammatory phases, which typically occur from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts. In the proliferative phase, which typically occurs from two days to three weeks, granulation tissue forms, and epithelialization and contraction begin. Fibroblasts, which are key cell types in this phase, proliferate and synthesize collagen to fill the wound and provide a strong matrix on which epithelial cells grow. As fibroblasts produce collagen, vascularization extends from nearby vessels, resulting in granulation tissue. Granulation tissue typically grows from the base of the wound. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility, and are common in granulating wounds. In the remodeling phase, the final phase of wound healing which can take place from three weeks up to several years, collagen in the scar undergoes repeated degradation and re-synthesis. During this phase, the tensile strength of the newly formed skin increases.

However, as the rate of wound healing increases, there is often an associated increase in scar formation. Scarring is a consequence of the healing process in most adult animal and human tissues. Scar tissue is not identical to the tissue which it replaces, as it is usually of inferior functional quality. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion.

In contrast, normal skin consists of collagen fibers arranged in a basket-weave pattern, which contributes to both the strength and elasticity of the dermis. Thus, to achieve a smoother wound healing process, an approach is needed that not only stimulates collagen production, but also does so in a way that reduces scar formation.

The biophotonic compositions and methods of the present disclosure promote wound healing by promoting the formation of substantially uniform epithelialization; promoting collagen synthesis; promoting controlled contraction; and/or by reducing the formation of scar tissue. In certain embodiments, the biophotonic compositions and methods of the present disclosure may promote wound healing by promoting the formation of substantially uniform epithelialization. In some embodiments, the biophotonic compositions and methods of the present disclosure promote collagen synthesis. In some other embodiments, the biophotonic compositions and methods of the present disclosure promote controlled contraction. In certain embodiments, the biophotonic compositions and methods of the present disclosure promote wound healing, for example, by reducing the formation of scar tissue or by speeding up the wound closure process. In certain embodiments, the biophotonic compositions and methods of the present disclosure promote wound healing, for example, by reducing inflammation. In certain embodiments, the biophotonic composition can be used following wound closure to optimize scar revision. In this case, the biophotonic composition may be applied at regular intervals such as once a week, or at an interval deemed appropriate by the physician or by other health care providers.

The biophotonic composition may be soaked into a woven or non-woven material or a sponge and applied as a wound dressing. A light source, such as LEDs or waveguides, may be provided within or adjacent the wound dressing or the composition to illuminate the composition. The waveguides can be optical fibres which can transmit light, not only from their ends, but also from their body. For example, the waveguides may be made of polycarbonate or polymethylmethacrylate.

In some embodiments, the methods of the present technology comprise assaying the level of energy production in the tissue treated and non treated with the biophotonic treatment of the present technology. Such assessment may be performed in vivo, or in vitro, or ex vivo, in situ, corporeally, or extra corporeally. In some instances, the assessment may be performed on a tissue sample (e.g., biopsy). In some embodiments, the assays useful for determining or measuring the cellular parameters discussed herein are well-known in the art.

The present disclosure also provides kits for skin treatment. The kit may include a composition, as defined herein, together with one or more of a light source, devices for applying or removing the composition, instructions of use for the composition and/or light source.

In some embodiments, the composition comprises at least a first light-absorbing molecule in a gelling agent. The light-absorbing molecule may be present in an amount of between about 0.001% and about 0.1%, between about 0.05% and about 1%, between about 0.5% and about 2%, between about 1% and about 5%, between about 2.5% and about 7.5%, between about 5% and about 10%, between about 7.5% and about 12.5%, between about 10% and about 15%, between about 12.5% and about 17.5%, between about 15% and about 20%, between about 17.5% and about 22.5%, between about 20% and about 25%, between about 22.5% and about 27.5%, between about 25% and about 30%, between about 27.5% and about 32.5%, between about 30% and about 35%, between about 32.5% and about 37.5%, or between about 35% and about 40% per weight of the composition. In embodiments where the composition comprises more than one light-absorbing molecule, the first light-absorbing molecule may be present in an amount of between about 0.01% and about 40% per weight of the composition, and a second light-absorbing molecule may be present in an amount of between about 0.0001% and about 40% per weight of the composition.

In certain embodiments, the first light-absorbing molecule is present in an amount of between about 0.01-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the second light-absorbing molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the amount of light-absorbing molecule or combination of light-absorbing molecules may be in the amount of between about 0.05-40.0% per weight of the composition. In certain embodiments, the amount of light-absorbing molecule or combination of light-absorbing molecules may be in the amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.0% per weight of the composition. The composition may include an oxygen-releasing agent present in amount between about 0.01%-40%, between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, or between about 30%-40% by weight to weight of the composition. Alternatively, the kit may include the oxygen-releasing agent as a separate component to the light-absorbing molecule containing composition.

In some embodiments, the kit includes more than one composition, for example, a first and a second composition. The first composition may include the oxygen-releasing agent and the second composition may include the first light-absorbing molecule in the gelling agent. The first light-absorbing molecule may have an emission wavelength between about 400 nm and about 570 nm. The oxygen-releasing agent may be present in the first composition in an amount of between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, between about 30%-40% or between about 35%-45% by weight to weight of the first composition. The light-absorbing molecule may be present in the second composition in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In embodiments where the second composition comprises more than one light-absorbing molecule, the first light-absorbing molecule may be present in an amount of between about 0.01-40% per weight of the second composition, and a second light-absorbing molecule may be present in an amount of about 0.0001-40% per weight of the second composition. In certain embodiments, the first light-absorbing molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In certain embodiments, the second light-absorbing molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In certain embodiments, the amount of light-absorbing molecule or combination of light-absorbing molecules may be in the amount of about 0.05-40.0% per weight of the second composition. In certain embodiments, the amount of light-absorbing molecule or combination of light-absorbing molecules may be in the amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.0% per weight of the second light-absorbing molecule.

In some other embodiments, the first composition may comprise the first light-absorbing molecule in a liquid or as a powder, and the second composition may comprise a gelling composition for thickening the first composition. The oxygen-releasing agent may be contained in the second composition or in a third composition in the kit. In some embodiments, the kit includes containers comprising the compositions of the present disclosure. In some embodiments, the kit includes a first container comprising a first composition that includes the oxygen-releasing agent, and a second container comprising a second composition that includes at least one light-absorbing molecule. The containers may be light impermeable, air-tight and/or leak resistant. Exemplary containers include, but are not limited to, syringes, vials, or pouches. The first and second compositions may be included within the same container but separated from one another until a user mixes the compositions. For example, the container may be a dual-chamber syringe where the contents of the chambers mix on expulsion of the compositions from the chambers. In another example, the pouch may include two chambers separated by a frangible membrane. In another example, one component may be contained in a syringe and injectable into a container comprising the second component. The composition may also be provided in a container comprising one or more chambers for holding one or more components of the composition, and an outlet in communication with the one or more chambers for discharging the composition from the container. In some embodiments, the kit comprises a systemic or topical drug for augmenting the treatment of the composition. For example, in certain such embodiments, the kit may include a systemic or topical agent, e.g., an anesthetics or anti-inflammation agent, for reducing pain.

Written instructions on how to use the composition in accordance with the present disclosure may be included in the kit, or may be included on or associated with the containers comprising the compositions of the present disclosure.

In certain embodiments, the kit may comprise a further component which is a dressing. The dressing may be a porous or semi-porous structure for receiving the composition. The dressing may comprise woven or non-woven fibrous materials.

In certain embodiments of the kit, the kit may further comprise a light source such as a portable light with a wavelength appropriate to activate the light-absorbing molecule in the composition. The portable light may be battery operated or re-chargeable.

In certain embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Assessing Progression of Wound Healing

A biophotonic regimen in accordance with one embodiment of the present technology was applied to wounds of several canine subjects (e.g., surgical wounds from orthopedic (joint), wounds from neurological surgeries or wounds created by traumatic ulcers) to assess the progression of healing of the wounds (i.e., assess tissue regeneration) treated with the biophotonic regimen compared to the progression of healing of the wounds not treated with the biophotonic regimen.

From the first day after surgery (T0) and then every 3 days (T1, T2, T3) until Day 13 (T4), 50% of the length of the surgical wound was treated with biophotonic therapy using a biophotonic composition. The biophotonic composition comprised a carrier gel comprising peroxide in the form of urea peroxide (UP) at a concentration of 3% w/w and eosin Y as a light-absorbing molecule-containing gel at a concentration of 0.01% w/w. The biophotonic membrane comprised eosin Y at a concentration of 0.08% w/w and 0% UP. The applied biophotonic composition/membrane was illuminated for a period of 2 minutes with a light source (KT-L™ Lamp, KLOX Technologies™ Inc., Laval, Canada) located at 5 cm from the wound (treated skin). The other 50% of the length of the surgical wound was treated with sterile saline (non-treated skin or control). The subjects were treated with the biophotonic therapy every 3 days until healing occurred. Every three days, the treatment area was cleaned with sterile isotonic saline and then treated with the biophotonic composition. After treatment, the wound was cleaned and then covered with a three-layer bandage. The surgical procedure, including the skin suture, was performed by the same orthopedic surgeon. Simple interrupting monofilament not absorbable suture was used to close the wound. At the end of treatment (Day 13) stitches were removed and 2 small biopsies (2 mm in diameter and 3-5 mm deep) were obtained in the median portions of the treated and control sides of the wound. No sutures were required. The evaluation protocol consisted of: a visual clinical scale (modified ASEPSIS scale) at T0, T1, T2 T3 and T4; histologic score at 13 days after surgery (see Table 1 for scoring system); immunohistochemistry analysis at 13 days after surgery to investigate expression of key cytokines and proteins involved in the wound healing process (TGFβ, TNFα, FVIII, FGF, EGF, Decorin, Collagen III, Ki67) (see Table 2 for results). The clinician that carried out the visual clinical score and the histopathologist that performed the microscopic examinations were blinded.

Fibroplasia begins 3-5 days after injury and may last as long as 14 days. Skin fibroblasts and mesenchymal cells differentiate to perform migratory and contractile capabilities. Fibroblasts migrate and proliferate in response to fibronectin, PDGF, fibroblast growth factor (FGF), transforming growth factor (TGFs), and C5a. Fibronectin serves as an anchor for the myofibroblast as it migrates within the wound. The synthesis and deposition of collagen is a critical event in the proliferative phase and to wound healing in general. Collagen is rich in hydroxylysine and hydroxyproline moieties, which enable it to form strong cross-links The hydroxylation of proline and lysine residues depends on the presence of oxygen, vitamin C, ferrous iron, and ketoglutarate. Deficiencies of oxygen and vitamin C, in particular, result in underhydroxylated collagen that is less capable of forming strong cross-links and, therefore, is more vulnerable to breakdown. Approximately 80% of the collagen in normal skin is type I collagen; the remaining is mostly type III. In contrast, type III collagen is the primary component of early granulation tissue and is abundant in embryonic tissue. Collagen fibers are deposited in a framework of fibronectin. An essential interaction seems to exist between fibronectin and collagen; experimental wounds depleted of fibronectin demonstrate decreased collagen accumulation. Elastin is also present in the wound in smaller amounts. Elastin is a structural protein with random coils that allow for stretch and recoil properties of the skin.

During remodeling, collagen improves in its organization. Fibronectin gradually disappears, and hyaluronic acid and glycosaminoglycans are replaced by proteoglycans. Type III collagen is replaced by type I collagen. Remodeling begins approximately 21 days after injury, when the net collagen content of the wound is stable. Cytokines have emerged as important mediators of wound healing events. The principal cytokines investigated were as follows: Epidermal growth factor (EGF), the first cytokine described as a potent mitogen for epithelial cells, endothelial cells, and fibroblasts. EGF stimulates fibronectin synthesis, angiogenesis, fibroplasia, and collagenase activity. Fibroblast growth factor (FGF), a mitogen for mesenchymal cells, represents an important trigger for angiogenesis (FGF is a mitogen for endothelial cells), fibroblasts, keratinocytes, and myoblasts. This factor also stimulates wound contraction and epithelialization and production of collagen, fibronectin, and proteoglycans. Transforming growth factor-beta (TGF-beta), released from the alpha granules of platelets and important stimulant for fibroblast proliferation and the production of proteoglycans, collagen, and fibrin. The factor also promotes accumulation of the extracellular matrix and fibrosis. Transforming growth factor-beta has been demonstrated to reduce scarring and to reverse the inhibition of wound healing by glucocorticoids. Tumor necrosis factor-alpha (TNF-alpha), produced by macrophages and stimulating angiogenesis and the synthesis of collagen and collagenase. It is a mitogen for fibroblasts. Decorin: an important proteoglycan involved in regulating collagen fibrillogenesis, and in interaction with other growth factors regulating their action, including CTGF. Proteoglycans play a role in cell signaling and can interact and modulate proteins found in the extracellular matrix. Decorin is known to bind to the three TGF-beta isoforms and to inhibit their activity by sequestering the isoforms to the extracellular matrix. Both fibromodulin and decorin have been shown to have lower levels or delayed expression in post-burn hypertrophic scars. This low or reduced expression may explain the irregular collagen organization and increased extracellular matrix production in pathological scarring. Decorin plays a role in reducing hypertrophic fibroblast proliferation, collagen synthesis and collagen contraction, inhibiting both basal and TGF-beta enhanced contraction in both normal and hypertrophic scar fibroblast.

TABLE 1
Interstitial Histologic Scoring System

| Feature Scored | Score | Description |
|---|---|---|
| Inflammation | 0 | None |
| Severity | 1 | Mild |
| | 2 | Moderate |
| | 3 | Severe |
| Inflammation | 0 | None |
| Extent | 1 | Epidermal |
| | 2 | Dermal and epidermal |
| | 3 | Transmural |
| Adnexa | 0 | None |
| Damage | 1 | 1/3 of adnexa damaged |
| | 2 | 2/3 of adnexa damaged |
| | 1 | Adnexa lost, surface |
| | | epithelium present |
| | 4 | Adnexa and surface |
| | | epithelium lost |
| Percent | 0 | 0% |
| Involvement | 1 | 1-25% |
| | 2 | 26-50% |
| | 3 | 51-75% |
| | 4 | 76-100% |

TABLE 2
Histological and immonohistochemistry of treated tissue

| | Control/ | | | | | | | | | |
|---|---|---|---|---|---|---|---|---|---|---|
| | treated | Histology | TGF β | TNF-α | FVIII | FGF | EGF | Decorin | Collagen III | Ki67 |
| | Score | Posit | Posit | Posit | Posit | Posit | Posit | Posit | Posit | Posit |
| Subject | Total | cells/field | cells/field | cells/field | cells/field | cells/field | cells/field | cells/field | cells/field | cells/field |
| 1 | Control | 14 | 2 | 282 | 11 | 27 | 4 | 22 | 305 | 68 |
| | Treated | 10 | 6 | 84 | 294 | 78 | 8 | 265 | 386 | 188 |
| 2 | Control | 13 | 5 | 97 | 103 | 34 | 108 | 21 | 83 | 282 |
| | Treated | 13 | 18 | 215 | 171 | 99 | 149 | 23 | 186 | 398 |
| 3 | Control | 11 | 68 | 275 | 59 | 423 | 43 | 76 | 98 | 253 |
| | Treated | 7 | 23 | 118 | 320 | 270 | 170 | 113 | 203 | 470 |
| 4 | Control | 8 | 40 | 178 | 220 | 292 | 35 | 86 | 80 | 143 |
| | Treated | 4 | 14 | 10 | 49 | 29 | 71 | 93 | 52 | 326 |
| 5 | Control | 9 | 9 | 258 | 252 | 81 | 21 | 96 | 125 | 161 |
| | Treated | 3 | 44 | 79 | 342 | 85 | 80 | 245 | 313 | 314 |
| 6 | Control | 13 | 13 | 137 | 41 | 24 | 38 | 28 | 89 | 218 |
| | Treated | 0 | 1 | 3 | 8 | 16 | 13 | 35 | 75 | 125 |
| 7 | Control | 14 | 93 | 204 | 96 | 343 | 9 | 49 | 76 | 171 |
| | Treated | 9 | 58 | 41 | 325 | 58 | 60 | 110 | 201 | 295 |
| 8 | Control | 13 | 13 | 130 | 56 | 34 | 15 | 15 | 86 | 63 |
| | Treated | 0 | 2 | 4 | 70 | 16 | 43 | 205 | 98 | 160 |
| 9 | Control | 9 | 15 | 205 | 80 | 120 | 5 | 21 | 70 | 200 |
| | Treated | 4 | 5 | 53 | 270 | 80 | 60 | 160 | 160 | 340 |
| 10 | Control | 13 | 40 | 190 | 76 | 102 | 21 | 15 | 81 | 140 |
| | Treated | 5 | 14 | 51 | 356 | 33 | 63 | 193 | 201 | 223 |

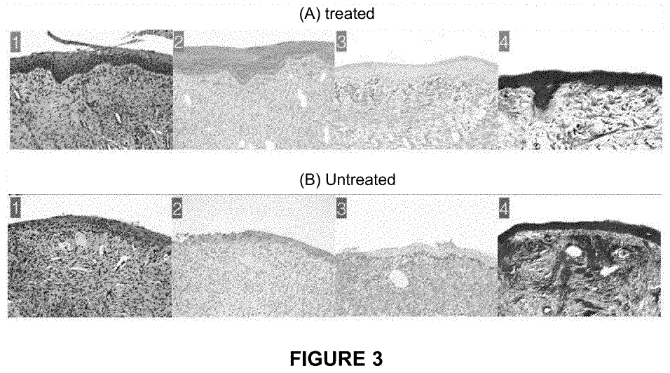

Statistical analysis was carried out on the data comparing treatment to control in a paired t-test and Wilcoxon signed rank test. For a valid paired t-test the data passed tests for both skewness and kurtosis and for a valid Wilcoxon test the data passed the test for skewness. Ten dogs of a variety of ages (from 1 y to 10 yrs) and breeds were prospectively recruited as they underwent orthopedic surgeries (5 TPLO, 3 limb alignments, 2 FHO). Ten incisional wounds and 20 biopsy samples (10 from treated area and 10 from control area) were examined No patient showed any adverse reaction to the treatment. Visual clinical assessment of the skin wounds revealed a better wound healing process at the treated area, with reduced scarring and minimal inflammation. No statistically significant differences were found. The biophotonic treated side had statistically significant better histology scores (p=0.001) showing better and more complete re-epithelialization, lesser inflammation of the dermal layer, less neo-angiogenesis and the presence of synthesis activities of the connective matrix (FIG. 3). A1 exhibits greater epidermal integrity and greater basal activity (greater dermic papillary fever) in the absence of residual flogosis and less neo-angeogenesis than in sample B1. Note the strong cytokeratinic expression of the A2 sample compared to the B2 sample, which, among other things, denotes a complete epidermal integrity in A2 compared to a partial re-epithelialization in B2. The expression of Collagen III in A3 is abundant compared to the B3 sample in which this expression is poor. A better and above all physiological repair is observed in A4 compared to B4; in A4 there is a greater and more regular deposition, compared to greater flogosis, blood extravasation and fibrosclerotic processes in B4 (blue tendency to black). Immunohistochemistry results showed, in the treated wound portions, a greater expression of FVIII (p=0.034), EGF (p=0.008), Decorin (p=0.005), Collagen III (p=0.005), Ki67 (p=0.002) and a lower expression of TGFβ (p<0.16), TNFα (p<0.001), FGF (p<0.098) (Table 2). These results indicate that the biophotonic regimen could also potentially represent a novel wound care technology, which utilizes fluorescence biomodulation to treat wounds by stimulating critical cellular pathways.

### Example 2—Assessing Mitochondrial Biogenesis in Tissue Undergoing Biophotonic Regimen

A biophotonic regimen in accordance with one embodiment of the present technology was applied to a skin region of canine subjects afflicted with type I or type IV immune-mediated dermatitis as well as with pyoderma to assess mitochondrial biogenesis in skin undergoing biophotonic regimen compared to mitochondrial biogenesis in skin not undergoing biophotonic regimen. From the first day after initial examination (T0) and then twice a week thereafter, a portion of the afflicted skin was treated with biophotonic therapy using a biophotonic composition or using a biophotonic membrane. The biophotonic composition comprised a carrier gel comprising peroxide in the form of urea peroxide (UP) at a concentration of 6% w/w and eosin Y as a light-absorbing molecule-containing gel at a concentration of 0.01% w/w. The biophotonic membrane comprised eosin Y at a concentration of 0.08% w/w and 0% UP. The applied biophotonic composition/membrane was illuminated for a period of 2 minutes with a light source (KT-L™ Lamp, KLOX Technologies™ Inc., Laval, Canada) located at 5 cm from the skin (treated skin). Another portion of the afflicted skin was treated with sterile saline (non-treated skin or control). The subjects were treated with the biophotonic therapy every 3 days until healing occurred. Every three days, the treatment area was cleaned with sterile isotonic saline and then treated with the biophotonic composition/membrane. The therapy was suspended after complete healing of the skin (T1) which occurred between 3 to 6 weeks following T0. Samples of skin from the areas treated with the biophotonic regimen and from the areas that were not treated with the biophotonic regimen were obtained at time (T0) and at (T1). The samples were assessed for the number and size of mitochondria via transmission electron microscopy. The results are presented in Table 3.

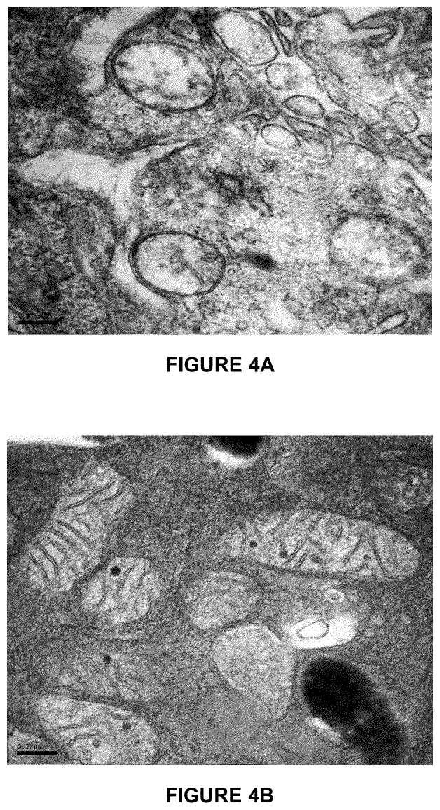

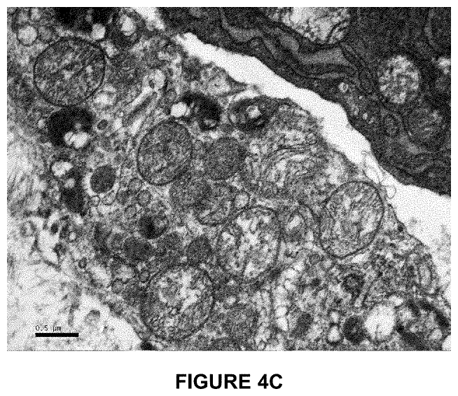

The morphology of the mitochondria was also assessed by electron microscopy. The tissue samples were prepared in accordance with standard tissue fixation procedures for transmission electron microscopy and embedded in resin. Fixed, embedded tissue pieces were thin-sectioned using an ultramicrotome and the sections were collected on copper grids. Sections were examined in a transmission electron microscope operating at 60-80 kV. FIGS. 4A, 4B and 4C show the morphology of the mitochondria in treated tissues before the commencement of the biophotonic treatment (T0) (FIG. 4A) and after completion of the biophotonic treatment (T1) (FIG. 4B) in comparison with non-treated tissue at T1 (FIG. 4C).

TABLE 3
Average mitochondrial counts and size in the cells of the treated tissue

| | | | | % Difference | Average | | |
|---|---|---|---|---|---|---|---|
| | | | Average # | in # | Size | % Difference | # Weeks |
| Subject | Time | Case # | Mitochondria | Mitochondria | (μm) | in Size (μm) | for healing |
| 11 | T0 | Ctr | 5.63 | 39 | 0.45 | 74 | 4 |
| | | TR | 3.5 | | 0.56 | | |
| | T1 | Ctr | 3.8 | | 0.98 | | |
| | | TR | 9.9 | 96 | 1.43 | 87 | |
| 12 | T0 | Ctr | 4.73 | 1 | 1.05 | 20 | 8 |
| | | TR | 4.33 | | 0.39 | | |
| | T1 | Ctr | 4.7 | | 0.86 | | |
| | | TR | 12.83 | 99 | 2.35 | 143 | |
| 13 | T0 | Ctr | 4.96 | 17 | 1.13 | 718 | 4 |
| | | TR | 5.5 | | 1.25 | | |
| | T1 | Ctr | 4.2 | | 0.95 | | |
| | | TR | 13.5 | 84 | 2.59 | 70 | |
| 14 | T0 | Ctr | 3.93 | 11 | 0.73 | 92 | 6 |
| | | TR | 4.77 | | 0.32 | | (dermatitis |
| | T1 | Ctr | 4.39 | | 1.98 | | and |
| | | TR | 9.44 | 66 | 1.98 | 144 | very deep |
| | | | | | | | pyoderma) |
| 15 | T0 | Ctr | 2.91 | 48 | 1.15 | 34 | 4 |
| | | TR | 3.22 | | 0.98 | | |
| | T1 | Ctr | 5.29 | | 0.82 | | |
| | | TR | 12.14 | 116 | 2.33 | 82 | |
| 16 | T0 | Ctr | 2.98 | 65 | 1.32 | 21 | 4 |
| | | TR | 2.5 | | 0.96 | | |
| | T1 | Ctr | 5.84 | | 1.07 | | |
| | | TR | 12.3 | 132 | 1.96 | 68 | |
| 17 | T0 | Ctr | 3.99 | 26 | 0.33 | 77 | 4 |
| | | TR | 5.1 | | 0.21 | | |
| | T1 | Ctr | 3.06 | | 0.74 | | |
| | | TR | 11.76 | 79 | 2.56 | 170 | |
| 18 | T0 | Ctr | 3.53 | 17 | 1.26 | 30 | 4 |
| | | TR | 5.36 | | 1.34 | | (deep |
| | T1 | Ctr | 2.99 | | 0.93 | | pyoderma |
| | | TR | 7.13 | 28 | 1.52 | 13 | and fleas |
| | | | | | | | (stimulate |
| | | | | | | | the |
| | | | | | | | immune |
| | | | | | | | system |
| | | | | | | | of the dog) |
| 19 | T0 | Ctr | 3.26 | 51 | 1.43 | 4 | 4 |
| | | TR | 3.26 | | 0.79 | | |
| | T1 | Ctr | 5.5 | | 1.37 | | |
| | | TR | 13.59 | 123 | 2.55 | 105 | |
| 20 | T0 | Ctr | 4.72 | 1 | 1.03 | 112 | 4 |
| | | TR | 6.16 | | 0.95 | | (deep |
| | T1 | Ctr | 4.79 | | 0.29 | | pyoderma |
| | | TR | 8.99 | 37 | 0.88 | 8 | and |
| | | | | | | | immune |
| | | | | | | | mediated |
| | | | | | | | dermatitis) |
| 21 | T0 | Ctr | 3.53 | 17 | 1.26 | 30 | 4 |
| | | TR | 5.36 | | 1.34 | | (deep |
| | T1 | Ctr | 2.99 | | 0.93 | | pyoderma |
| | | TR | 7.13 | 28 | 1.52 | 13 | and |
| | | | | | | | immune |
| | | | | | | | mediated |
| | | | | | | | dermatitis) |
| 22 | T0 | Ctr | 2.83 | 52 | 0.58 | 94 | 3 |
| | | TR | 2.9 | | 0.62 | | |
| | T1 | Ctr | 4.8 | | 1.61 | | |
| | | TR | 11.89 | 122 | 2.57 | 122 | |
| Control | T0 | Ctl | 2.45 | 56.30 | 0.98 | 6.68 | |
| | | TR | 4.36 | | 1.04 | | |
| | T1 | Ctl | 4.33 | | 0.81 | | |
| | | TR | 10.88 | 86.15 | 2.02 | 85.60 | |

T0 = start of the phototherapy; and T1 = closure of the wound

These data show that number of mitochondria increased in the tissue treated with the biophotonic regimen compared to the tissues that were not treated. The results suggest that biophotonic treatment contributes to stimulate mitochondrial biogenesis.

### Example 3—Assessing ATP Levels

To further assess the efficiency of a biophotonic regimen in accordance with the present technology at, for example, healing of skin disorders, an assay will be performed to determine the levels of cellular ATP production by cells of treated tissue/skin during the course of the biophotonic regimen. Samples of skin/tissue as well as samples of non-treated skin/tissues will be obtained prior to the commencement of the biophotonic regimen and additional samples will be obtained at different time points during the course of the biophotonic regimen. The samples will be assessed for the levels of ATP production. ATP is a molecule found only in and around living cells, and as such it gives a direct measure of biological concentration and health. ATP may be quantified by measuring the light produced through its reaction with the naturally-occurring firefly enzyme Luciferase using a Luminometer. The amount of light produced is directly proportional to the amount of biological energy present in the sample. Levels of ATP production may also be measured using one or more of the following techniques: ATP colorimetric/fluorometric assay, ATP luminescence assay, ATP immonochemistry assay.

### Example 4—Measurement of Respiration

To further assess the efficiency of a biophotonic regimen in accordance with the present technology at, for example, healing of skin disorders, an assay will be performed to measure the level of respiration in cells of treated tissue/skin. Oxygen consumption by intact cells will be measured as an indication of mitochondrial respiration activity. The BD Oxygen Biosensor System (BD Biosciences, Franklin Lakes, N.J., USA) is an oxygen sensitive fluorescent compound (tris 1,7-diphenyl-1,10 phenanthroline ruthenium (II) chloride) embedded in a gas permeable and hydrophobic matrix permanently attached to the bottom of a multiwell plate. The concentration of oxygen in the vicinity of the dye is in equilibrium with that in the liquid media. Oxygen quenches the dye in a predictable concentration dependent manner The amount of fluorescence correlates directly to the rate of oxygen consumption in the well, which in turn can relate to any sort of reaction that can be linked to oxygen consumption. The unique technology allows homogenous instantaneous detection of oxygen levels. After treatment, cells will be were washed in KRH buffer plus 1% BSA. Cells from each condition will be divided into aliquots in a BD Oxygen Biosensor System plate (BD Biosciences) in triplicate. Plates will be sealed and “read” on a Fluorescence spectrometer (Molecular probes) at 1-minute intervals for 60 minutes at an excitation wavelength of 485 nm and emission wavelength of 630 nm.

### Example 5—Measurement of Mitochondrial DNA

To further assess the efficiency of a biophotonic regimen in accordance with the present technology at, for example, healing of skin disorders, an assay will be performed to measure the levels of mitochondrial DNA in the cells of treated tissue/skin. Quantitative PCR will be performed in Mx3000P Real-Time PCR system (Stratagene). Reactions will be performed with 12.5 microliters SYBR-Green Master Mix (ABI), 0.5 microliters of each primer (10 microM), 100 ng template (DNA) or no template (NTC), and RNAse-free water was added to a final volume of 25 microliters. Each quantitative PCR will be performed in triplicate. The following primers will be used: mitochondrial D-loop forward, mitochondrial D-loop reverse, 18SRNA forward, and 18SRNA reverse. The mouse 18S rRNA gene will serve as the endogenous reference gene. A melting curve will be done to ensure specific amplification. The standard curve method will be used for relative quantification. The ratio of mitochondrial D-loop to 18S rRNA will then be calculated. Final results will be presented as percentage of control.

### Example 6—Assays for Activities of Mitochondrial Complex I, II, and III

To further assess the efficiency of a biophotonic regimen in accordance with the present technology at, for example, healing of skin disorders, an assay will be performed to evaluate the activity level of mitochondria Complex I, II and III in cells of treated tissue/skin. Treated cells will be cultured in 100 mm plates, washed in PBS, resuspended in an appropriate isotonic buffer (0.25 M sucrose, 5 mM Tris-HCl, pH 7.5, and 0.1 mM phenylmethylsulfonyl fluoride), and homogenized. Mitochondria will be isolated by differential centrifugation of the cell homogenates. NADH-CoQ oxidoreductase (Complex I), succinate-CoQ oxidoreductase (complex II), CoQ-cytochrome c reductase (complex III) will be assayed spectrometrically using the conventional assays (Picklo and Montine, 2001 Biochim Biophys Acta 1535: 145-152; Humphries, K. M., and Szweda, L. I. 1998 Biochemistry 37:15835-15841), with minor modifications.

### Example 7—Assays for Expression of Mitochondrial Enzyme COX IV

An assay was performed to evaluate the levels of expression of mitochondria enzyme COX-IV in swine skin samples injected with the biophotonic compositions of the present technology and exposed to a biophotonic treatment according to one embodiment of the present disclosure. Cytochrome c oxidase or complex IV (COX IV), catalyzes the final step in mitochondrial electron transfer chain, and is regarded as one of the major regulation sites for oxidative phosphorylation. This enzyme is controlled by both nuclear and mitochondrial genomes. Among its 13 subunits, three are encoded by mitochondrial DNA and ten by nuclear DNA. A detail biosynthetic and functional analysis of several cell lines with suppressed COX IV expression revealed a loss of assembly of cytochrome c oxidase complex and, correspondingly, a reduction in cytochrome c oxidase-dependent respiration and total respiration. Furthermore, dysfunctional cytochrome c oxidase in the cells leads to a compromised mitochondrial membrane potential, a decreased ATP level, and failure to grow in galactose medium. Interestingly, suppression of COX IV expression also sensitizes the cells to apoptosis. These observations provide the evidence of the essential role of the COX IV subunit for a functional cytochrome c oxidase complex and also demonstrate a tight control of cytochrome c oxidase over oxidative phosphorylation.

The biophotonic compositions were prepared as follows: a 1 cc syringe was filled with a tissue filler composition and another 1 cc syringe was filled with the light-absorbing molecule composition. The content of the two syringes were mixed together via a connector right before injection into the skin of the pigs. The light-absorbing molecules and tissue fillers used in the preparation of the compositions were as follows: i) 0.012% of Eosin Y; ii) 0.012% of Eosin Y and Fluorescein. Tissue filler selected from: i) Emervel® Classic (Galderma, Lausanne, Switzerland); ii) Emervel® Volume (Galderma, Lausanne, Switzerland); iii) Radiesse® (Merz Aesthetics, NC, USA).

Each animal was placed in ventral recumbency. The hair was removed from the treatment area on the back of the animal. The surgical site was prepared with topical cleaning using a neutral (non-antibacterial nor antiseptic) soap, rinsed with sterile saline followed by an application of 70% isopropyl alcohol. Ten areas were drawn with a skin marker or tattooed to delineate the sites of injection and incision. Klox Thera® lamp was used for the illumination of the injected skin section and activation of the biophotonic composition injected therein. Specifically, Klox Thera® lamp with Blue LEDs (B LED) and Klox Thera® lamp with Green LEDs (G LED) were used on the injected samples. The wavelengths of the green or blue light emitted ranged between 420 nm to 490 nm or with a wavelength around 566 nm. The irradiance or power density of the light was between 100 mW/cm² and 150 mW/cm² at a distance of 5 cm from the light source with a radiant fluences (or dose) during a single treatment for 5 minutes of 33 J/cm² to 45 J/cm². Forty-six subcutaneous injections (100 μl to 300 μl each) were performed. Four skin incisions of ˜8 cm length were performed on each animal Each incision were rinsed with sterile saline and dried with sterile gauze. The incisions were then be sutured using 4-0 Ethicon. The pig modes used for the study were as defined in Table 4.

TABLE 4
characteristics of animal model

| Species: | Sus scrofa |
|---|---|
| Strain: | Hybrid farm pigs |
| | (Landrace-Yorkshire) |
| Condition: | Non diseased |
| Source: | Triporc Inc. |
| Age at implant: | Young adult |
| Weight at implant: | 20 ± 5 kg |
| Sex: | Female |
| Number: | 12 + 1 spares |
| Strain: | Hybrid farm pigs |
| | (Landrace-Yorkshire) |

Samples of skin from the injected areas were stained by the using of specific monoclonal antibody for detection of COX IV in sections (The antibody: 100 μL COX4 Antibody (Monoclonal, 6B3): 100 μL COX4 Antibody (Monoclonal, GT6310) 1 mg/ml COX4 Antibody (Monoclonal, GT6310). Host Mouse; Target Species Human, Rat, Swine. Unconjugated. Catalog # MA517279. Size 100 μL). The antibody is applicable in paraffin-embedded tissues for Immunocytochemistry (1:100-1:1000). Positive cells were counted. The data presented in Table 5 shows the effect of varying the type of light-absorbing molecules and light on COX IV expression levels. The data obtained demonstrates that skin sections injected with the biophotonic compositions comprising comprising Eosin Y or Eosin Y/Fluorescein as light-absorbing molecule(s) and exposed to blue or green light showed increase synthesis of COX VI after the phototreatment.

TABLE 5
Levels of COX IV expression in skin treated with
biophotonic compositions

| | | COX IV |
|---|---|---|
| | | (POS |
| Tissue | | (cells/ |
| Filler | Biophotonic Composition and Phototreatment | field) |
| Emervelle | Emervelle Classique, Eosin, Blue Light | 171 |
| | Emervelle Classique, Eosin, Green Light | 364 |
| | Emervelle Classique, Eosin, No Light | 33 |
| | Emervelle Classique, Eosin/Fluorescein, Blue Light | 386 |
| | Emervelle Classique, Eosin/Fluorescein, Green Light | 483 |
| | Emervelle Classique, Eosin/Fluorescein, No Light | 31 |
| | Emervelle Classique, Placebo, Blue Light | 184 |
| | Emervelle Classique, Placebo, Green Light | 263 |
| | Emervelle Classique, Placebo, No Light | 48 |
| | Emervelle Volume, Eosin, Green Light | 406 |
| | Emervelle Volume, Eosin/Fluorescein, Green Light | 564 |
| | Emervelle Volume, Placebo, Green Light | 103 |
| | No Injection, Placebo, Green Light | 88 |
| | Placebo, Placebo, Blue Light | 15 |
| | Placebo, Placebo, No Light | 9 |
| Radiesse | Radiesse, Eosin, Blue Light | 144 |
| | Radiesse, Eosin, Green Light | 204 |
| | Radiesse, Eosin, No Light | 43 |
| | Radiesse, Eosin/Fluorescein, Blue Light | 206 |
| | Radiesse, Eosin/Fluorescein, Green Light | 303 |
| | Radiesse, Eosin/Fluorescein, No Light | 29 |
| | Radiesse, Placebo, Blue Light | 92 |
| | Radiesse, Placebo, Green Light | 132 |
| | Radiesse, Placebo, No Light | 41 |

These results suggest that different fluorescence emitted by the light-absorbing molecule may biomodulate some biological/biochemical processes. While the present technology has been described in connection with specific embodiments thereof, it will be understood that it is capable of further modifications and this application is intended to cover any variations, uses, or adaptations of the invention following, in general, the principles of the present technology and including such departures from the present disclosure as come within known or customary practice within the art to which the present technology pertains and as may be applied to the essential features hereinbefore set forth, and as follows in the scope of the appended claims.

1. A method for modulating a biophotonic treatment of a subject, the method comprising:
a) assaying a level of energy production in tissue of the subject under biophotonic treatment;
b) comparing the level of energy production assayed in a) with a level of energy production in non-treated tissue, and
c) wherein when the level of energy production obtained in a) is lower than the level of energy production obtained in b), varying the parameters of the biophotonic treatment.

2. The method as defined in claim 1, wherein the non-treated tissue is from the subject undergoing the biophotonic treatment.

3. The method as defined in claim 1, wherein the biophotonic treatment comprises:
i) applying a biophotonic composition to the tissue; and
ii) illuminating the applied biophotonic composition with a light emitted from a light source for a time sufficient to activate the biophotonic composition.

4. The method as defined in claim 3, wherein the parameters of the biophotonic treatment are selected from one or more of: wavelength of the light, power density of the light, fluence of the light, duration of illumination, distance between the light source and the tissue, frequency of the biophotonic treatment, and composition of the biophotonic composition.

5. The method as defined in claim 1, wherein the biophotonic composition comprises at least one light-absorbing molecule.

6. The method as defined in claim 5, wherein the at least one light-absorbing molecule is selected from a xanthene dye, a xanthene derivative dye, an azo dye, a biological stain, and a carotenoid.

7. The method as defined in claim 5, wherein the at least one light-absorbing molecule is selected from eosin, erythrosine, and fluorescein.

8. The method as defined in claim 7, wherein the at least one light-absorbing molecule is eosin Y or eosin B.

9. The method as defined in claim 8, wherein the at least one light-absorbing molecule is eosin Y.

10. The method as defined in 8, wherein the at least one light-absorbing molecule is erythrosine B.

11. The method as defined in claim 5, wherein the at least one light-absorbing molecule is an endogenous light absorbing-molecule.

12. The method as defined in claim 11, wherein the endogenous light absorbing-molecule is a vitamin B.

13. The method as defined in claim 11, wherein the endogenous light absorbing-molecule is 7,8-Dimethyl-10-[(2S,3S,4R)-2,3,4,5-tetrahydroxypentyl]benzo[g]pteridine-2,4-dione.

14. The method as defined in claim 3, wherein the at least one light-absorbing molecule is present in an amount of at least about 0.01% by weight of total volume of the biophotonic composition.

15. The method as defined in claim 1, wherein the step of assaying energy production includes measuring the level of at least one cellular marker associated with energy production.

16. The method as defined in claim 15, wherein the step of measuring the level of the at least one cellular marker associated with energy production is achieved by measuring the level of the at least one cellular marker in the tissue.

17. The method as defined in claim 15, wherein the at least one cellular marker associated with energy production is selected from ATP, ADP, GTP, GDP, ATP synthase, NADH, NAD, FAD, FADH, pyruvate, succinate, fumarate, co-enzyme A, pyruvate dehydrogenase, acetyl-CoA, citrate synthase, citrate, aconitase, isocitrate dehydrogenase, alpha-ketogluterate dehydrogenase, succinyl-CoA, succinyl CoC dehydrogenase, succinate dehydrogenase, fumarase, malate, malate dehydrogenase, oxalo acetate, citric acid, NADH-coenzyme Q oxidoreductase, succinate-Q oxidoreductase, flavoprotein-Q oxidoreductase, Q-cytochrome c oxidoreductase, cytochrome c and cytochrome c oxidase.

18. The method as defined in claim 15, wherein the at least one cellular marker associated with energy production is COX IV.

19.-32. (canceled)

33. A method for modulating efficiency of a biophotonic regimen at healing a wounded tissue; the method comprising:
a) measuring the level of ATP production in the wounded tissue prior to commencement of the biophotonic regimen;
b) measuring the level of ATP production in the wounded tissue after commencement of the biophotonic regimen; wherein when the level of ATP production in b) is lower than the level of ATP production in a), parameters of the biophotonic regimen are modulated.

34. A method for stimulating mitochondrial biogenesis in a tissue, the method comprising applying a biophotonic composition to the tissue; and illuminating the applied biophotonic composition for a time sufficient to activate the biophotonic composition.

35.-47. (canceled)
